(19) United States
(12) Patent Application Publication
(10) Pub. No.: US 2026/0327693 A1
(43) Pub. Date: Oct. 22, 2026

(54) AMPLICON BASED ROLLING CIRCLE AMPLIFICATION

(71) Applicant: ADOR DIAGNOSTICS LTD, Limassol (CY)

(72) Inventors: Vladimir HURGIN, Gan Yavne (IL); Anastasiya SHULMAN, Rehovot (IL); Porat HANANI, Tel Aviv (IL); Ronna SHAYEVITCH, Kfar Sava (IL); Alon Nissim BEN EFRAIM, Jerusalem (IL); Aya KHWAJA, Jatt (IL); Luba FARBEROV, Ashdod (IL); Giora VOLPERT, Kibbutz Ein Shemer (IL); Eran ZAHAVY, Rehovot (IL); Ido DROMI, Beit Nekofa (IL)

(21) Appl. No.: 19/483,523

(22) PCT Filed: Feb. 14, 2024
(86) PCT No.: PCT/IL2024/050172

§ 371 Date: Nov. 11, 2025

Related U.S. Application Data
(60) Provisional application No. 63/445,507, filed on Feb. 14, 2023.
(60) Provisional application No. 63/531,895, filed on Aug. 10, 2023.

(51) Int. Cl. C12Q 1/6855; C12Q 1/44; C12Q 1/48; C12Q 1/6806; C12Q 1/6813; C12Q 1/6888; C12Q 1/70

(52) U.S. Cl. CPC C12Q 1/6855; C12Q 1/44; C12Q 1/48; C12Q 1/6806; C12Q 1/6813; C12Q 1/6888; C12Q 1/70; C12Y 207/07

(57) ABSTRACT

Some embodiments are directed towards methods for detection of a target nucleic acid in a sample, including converting the target nucleic acid into an amplicon, for example using recombinase/polymerase amplification (RPA) or homologous recombination, further amplifying the amplicon by performing a rolling circle amplification (RCA) and detecting the final RCA product. The presently disclosed subject matter further provides a cartridge, a kit and a system for performing the detection methods.

### CROSS-REFERENCE TO RELATED APPLICATION

This application is a national phase filing under 35 C.F.R. § 371 of and claims priority to PCT Patent Application No. PCT/IL 2024/050172, filed on Feb. 14, 2024, which claims the benefit of U.S. Provisional Application No. 63/445,507, filed on Feb. 14, 2023, and of U.S. Provisional Application No. 63/531,895, filed on Aug. 10, 2023, the contents of which are hereby incorporated in their entireties by reference.

### TECHNOLOGICAL FIELD

The present disclosure concerns diagnostic assays for detection of target nucleic acids, specifically for rapid detection of multiple pathogens in clinical samples.

### BACKGROUND ART

References considered to be relevant as background to the presently disclosed subject matter are listed below:
1 Ali et al., “Rolling circle amplification: a versatile tool for chemical biology, materials science and medicine” Chem. Soc. Rev., (2014) 43, 3324-3341.
2. Glokler et al., “Isothermal amplifications—a comprehensive review on current methods” Crit. Rev. Biochem. Mol. Biol. (2021) 56 (6): 543-586.
3. Li et al., “Review: a comprehensive summary of a decade development of the recombinase polymerase amplification” Analyst, (2019), 144, 31.
4. Liu and Morrical, “Assembly and dynamics of the bacteriophage T4 homologous recombination machinery” Virol. J. (2010) 7:357.
5. Morrical et al., “Amplification of Snap-back. DNA synthesis reactions by the uvaX recombinase of bacteriophage T4” J. Biol. Chem. (1991) Vol. 266, No. 21, pp 14031-14038.
6. Murakami et al., “Sensitive isothermal detection of nucleic-acid sequence by primer generation-rolling circle amplification” Nucleic Acids Research (2009), Vol. 37, No. 3.
7. Piepenburg et al., “DNA detection using recombination proteins” PLoS Biol. (2006) 4 (7): e204.
8. Zhang and Tanner, “Isothermal amplification of long, discrete DNA, fragments facilitated by single-stranded binding protein” Sci. Rep. (2017) 7(1): 8497.

Acknowledgement of the above references herein is not to be inferred as meaning that these are in any way relevant to the patentability of the presently disclosed subject matter.

### BACKGROUND

The ability to identify rapidly and accurately the causative pathogen in a clinical sample is a critical step for applying suitable therapy. Such identification should be sensitive enough to detect low copy number of the pathogen and ideally be multiplexed such that multiple causative agents can be tested in parallel. Over modern human history, the world has repeatedly experienced pandemic outbreaks of infectious diseases, such as bubonic plague, smallpox, cholera, tuberculosis, influenza, Ebola, Zika and the very recent COVID-19 pandemic. One of the lessons learned during the COVID-19 pandemic is the importance of national and international preparedness to these situations, by having diagnostic tools prepared in advance to ensure an effective and immediate response to a new threat.

The introduction of nucleic acid amplification techniques has revolutionized the field of medical diagnostics in the last decade (Glokler et al.). Isothermal amplification is one such technique that accumulates nucleic acid sequences (amplicons) at constant temperature thus overcoming the constraints associates with the classic polymerase chain reaction (PCR) which requires a sophisticated thermocycling machine for its performance Isothermal amplification has been used for detecting DNA and RNA.

Among the most well-established isothermal amplification methods are Recombinase polymerase amplification (RPA), homologous recombination, and Rolling circle amplification (RCA).

The RPA concept, was originally described by Morrical et al., who showed that the replication-initiating activity of the uvsX protein greatly amplifies hairpin-primed DNA synthesis that is catalyzed by the T4 DNA polymerase holoenzyme on linear, single-stranded DNA templates.

Piepenburg et al (2006) were the first to describe DNA detection methods using RPA. In RPA, the isothermal amplification of specific DNA fragments is achieved by the binding of opposing oligonucleotide primers to template DNA and their extension by a DNA polymerase. Briefly, recombinase/primer filaments scan template DNA for homologous sequences. Following strand exchange, the displaced strand is bound by the Gp32 ssDNA-binding protein (34 kDa), and primers are extended by Bsu polymerase. The net result of two opposing primer binding/extension events is the generation of one complete copy of the amplicon in addition to the original template. Repetition of the process results in exponential DNA amplification. The key to RPA is the establishment of a dynamic reaction environment that balances the formation and disassembly of recombinase-primer filaments. In the presence of ATP, uvsX (44 kDa) binds cooperatively to oligonucleotides. Upon ATP hydrolysis, the nucleoprotein complex disassembles and uvsX can be replaced by Gp32. The presence of uvsY and crowding agents shifts the equilibrium in favor of recombinase loading (Piepenburg et al. (2006)).

Presently, RPA is used as an isothermal molecular tool for the identification of pathogens. It has been applied in laboratory settings as well as in automated microfluidics devices (Li et al.).

Lio and Monical (2010) is a review describing the assembly and dynamics of the bacteriophage T4 homologous recombination (HR) machinery. HR is a process involving the physical exchange of strands between homologous or nearly homologous DNA molecules.

Bacteriophage T4 routinely generates ssDNA during replication of its linear chromosome ends. The production of ssDNA tails or gaps in otherwise duplex DNA allows the assembly of core recombination machinery including presynaptic filaments on ssDNA. Presynaptic filaments are helical nucleoprotein filaments consisting of a recombinase enzyme and its accessory proteins bound cooperatively to ssDNA. The presynapsis pathway in bacteriophage T4 homologous recombination generally comprises the following steps: a dsDNA end is resected to expose a 3′ ssDNA tail probably by the exonucleases Gp46 and Gp47 proteins. The exposed ssDNA is sequestered by the Gp32 ssDNA-binding protein, which denatures secondary structure in ssDNA and keeps it in an extended conformation. The UvsY recombination mediator protein forms a tripartite complex with Gp32 and ssDNA and “primes” the complex for recruitment of UvsX recombinase which is ATP dependent and binds in a sequence independent manner to both ssDNA and dsDNA. UvsY recruits ATP-bound UvsX protein and nucleates presynaptic filament formation. Gp32 is displaced in the process (Liu and Morrical (2010)).

Zhang and Tanner (2017) describe isothermal amplification of DNA fragments facilitated by a single-stranded binding protein. They report the amplification of discrete target fragments from both double- and single-stranded circular template DNA. This amplification requires only the single-stranded DNA-binding protein gp32 from bacteriophage T4 and a strand-displacing DNA polymerase. Accordingly, T4 Gp32-primer complexes are generated. T4 Gp32 facilitates the primer strand invasion into double-stranded DNA allowing subsequent polymerase strand displacement activity. The duration of the protocol described by Zhang and Tanner is about 1 hour, it involves the identification of a single target pathogen (defined as “single-plex”) with a limit of detection of 3.5×10¹⁰.

RCA is an isothermal process where a short DNA or RNA primer is amplified to form a long single stranded DNA or RNA using a circular DNA probe and a DNA or RNA polymerase (Ali et al.). Murakarni et al. describe a mode of RCA dubbed primer generation RCA (PG-RCA). The RCA product is a concatemer containing tens to hundreds of tandem repeats that are complementary to the circular probe. It has been used, among others, to develop diagnostic methods for detecting nucleic acids.

Yet, as valuable as RPA and RCA are there is still room for improvement in sensitivity, multiplexity, confirmation of clinical relevance, speed of the reaction and applicability to on-site or field application. Therefore, despite the existing amplification methods for detection of target nucleic acids, there is still a need for a method that is accurate, fast, and capable of detecting low copy numbers of pathogens, particularly in clinical samples.

### GENERAL DESCRIPTION

In a first of its aspects, the present invention provides a method for detection of a target nucleic acid in a sample, comprising:
a. Converting said target nucleic acid into an amplicon;
b. Further amplifying said amplicon by performing a rolling circle amplification (RCA) to obtain an RCA product; and
c. Detecting said RCA product,
   Wherein a positive detection signal indicates the presence of said nucleic acid in the sample.

In one embodiment, the step of converting the target nucleic acid into an amplicon comprises isothermal amplification.

In one aspect, the present invention provides a method for detection of a target nucleic acid in a sample, comprising:
a. Obtaining nucleic acids, wherein said nucleic acids were extracted and eluted from said sample;
b. Amplifying target specific nucleic acid sequences by isothermal amplification with target specific primers;
c. Incubating the amplified target sequences obtained in step (b) with a sequence specific open circular probe and ligating the nick in the open circular probe;
d. Performing a rolling circle amplification (RCA) to obtain RCA products; and
e. Detecting said RCA product,
   Wherein a positive detection signal indicates the presence of the target nucleic acid in the sample.

In one embodiment, the method is for the detection of at least one pathogen in a sample.

In one embodiment, said nucleic acids are genomic DNA, or RNA.

In one embodiment, said nucleic acids were extracted by incubating the sample with a solution comprising a lysis reagent.

In one embodiment, the lysis reagent is Guanidinium Thiocyanate (GuTCN).

In one embodiment, said solution further comprises sodium N-lauroylsarcosinate and a protease.

In one embodiment, the nucleic acid elution is performed in a recombinase reaction buffer.

In one embodiment, said nucleic acid elution is performed by incubating the extracted nucleic acids in a buffer comprising at least one set of forward primer and reverse primer that hybridize to said target nucleic acid sequences, nucleoside triphosphates, magnesium acetate, optionally potassium acetate and optionally a hydrophilic polymer, e.g., polyethylene glycol (PEG).

In one embodiment, said incubation is for 2 min at 56° C.

In one embodiment, the isothermal amplification is recombinase polymerase amplification (RPA).

In one embodiment, the RPA is performed by incubating the eluted nucleic acids with at least one recombinase, at least one polymerase, and optionally at least one reverse transcriptase.

In one embodiment, said incubation is for between about 5 minutes and about 40 minutes, e.g., 20 minutes at a temperature of between about 37° C. and 42° C., e.g., 40° C.

In one embodiment, said at least one set of forward primer and reverse primer that hybridize to said target nucleic acid sequences are pathogen specific primers selected from a group consisting of SEQ ID Nos: 1-18, 47-52, 56 and 58.

In one embodiment, said isothermal amplification is performed by homologous recombination (HR).

In one embodiment, said homologous recombination is performed using a strand displacing DNA polymerase.

In one embodiment, said homologous recombination is performed by incubating the extracted nucleic acids with at least one strand displacing DNA polymerase, at least one set of forward primer and reverse primer that hybridize to said target nucleic acid sequences, nucleoside triphosphates, magnesium sulfate, optionally at least one set of outer forward primer and outer reverse primer, and optionally at least one reverse transcriptase.

In one embodiment, said strand displacing DNA polymerase is BST3 large fragment.

In one embodiment, said incubation is for between about 10 minutes and about 20 minutes, e.g., 15 minutes at a temperature of between about 60° C. and 70° C., e.g., 65° C.

In another embodiment, said homologous recombination is performed using a strand displacing DNA polymerase together with bacteriophage T4 Gp32.

In one embodiment, said homologous recombination is performed by incubating the extracted nucleic acids with bacteriophage T4 Gp32, at least one DNA polymerase, at least one set of forward primer and reverse primer that hybridize to said target nucleic acid sequences, nucleoside triphosphates, magnesium acetate, optionally potassium acetate and optionally at least one reverse transcriptase.

In one embodiment, said incubation is for between about 5 minutes and about 15 minutes, e.g., 10 minutes at a temperature of between about 37° C. and 65° C.

In one embodiment, said at least one set of forward primer and reverse primer that hybridize to said target nucleic acid sequences are pathogen specific primers selected from a group consisting of SEQ ID Nos: 29-46.

In one embodiment, said sequence specific open circular probe is a pathogen specific probe selected from a group consisting of SEQ ID Nos: 20-28, and 53-55.

In one embodiment, the method further comprises a step of exonuclease treatment after ligating the nick in the open circular probe and prior to performing the RCA.

In one embodiment, said RCA is performed using branching forward primers and branching reverse primers.

In one embodiment, the RCA is performed in the presence of nickase.

In one embodiment, said detecting the RCA product comprises:
a. Cutting said RCA product;
b. Addressing the cut RCA products onto a substrate comprising target specific capture probes;
c. Adding a target specific detectably labelled reporter complex; and
d Visualizing the captured RCA products.

In one embodiment, said method is implemented in a cartridge.

In one embodiment, the method is for multiplex identification of a panel of pathogens.

In one embodiment, each of said panel of pathogens is responsible for a disease selected from a group consisting of a respiratory disease, a cerebrospinal fluid infection, a sexually transmitted disease, tuberculosis, gastrointestinal diseases, drug resistant pathogens, plant pathogens, animal pathogens, and sepsis.

In another aspect, the present invention provides a cartridge for detecting at least one pathogen in a sample, comprising buffers, solutions, primers, probes and enzymes for performing:
a. Pathogen lysis;
b. Nucleic acid purification;
c. Target amplification;
d. Ligation of a circular probe with the amplified target;
e. RCA;
f. Digestion; and
g. Detection.

In another aspect, the present invention provides a kit for detecting at least one pathogen in a sample, comprising:
a. A cartridge according to the invention; and
b. Instructions for use.

In another aspect, the present invention provides a system for detecting at least one pathogen in a sample, comprising:
a. A benchtop analyzer; and
b. At least one cartridge according to the invention.

### BRIEF DESCRIPTION OF THE DRAWINGS

To exemplify and better understand the subject matter that is disclosed herein, embodiments will now be described, by way of non-limiting example only, with reference to the accompanying drawings, in which:

FIG. 1 shows RCA products detected using sensor imaging. The top panel shows the sensor design. The sensor comprises pathogen specific capture nucleic acids for: Chp (Chlamydia pneumonia), Bp (Bordetella pertussis), Mp (Mycoplasma pneumonia), RSV A (respiratory Syncytial virus A), RSV B (respiratory Syncytial virus B), Flu B (influenza B). Each of the lower panels represents results obtained with a sample of the indicated pathogen. Highlighted spots represent a positive result. The dots on the right and left sides represent positive control for the hydrogel integrity.

FIGS. 2A and 2B show gel analysis of RCA products for detection of B. pertussis. FIG. 2A: Lanes 1 to 4 were loaded with RCA products obtained from a sample containing 1×10³ copies of the pathogen, lanes 5 to 8 were loaded with RCA products obtained from a sample containing 1×10² copies of the pathogen. Lane 9: synthetic B. pertussis DNA. Lane 10: Non-specific sDNA (synthetic DNA). Lane M: (molecular weight (Mw) ladder). FIG. 2B: Lanes 1 to 4 were loaded with RCA products obtained from a sample containing 1×10¹ copies of the pathogen. Lane 5: synthetic B. pertussis DNA. Lane 6: Non-specific sDNA.

FIG. 2C-2D are graphs showing qPCR amplification curves (delta Rn) of the recombinase polymerase pre-amplification reactions as a function of reaction cycle. FIGS. 2C and 2D are duplicates. Three time points were analyzed as indicated by the arrows in the graph: time 0 (no incubation, light grey lines), time 30 (30 minutes incubation, grey lines) and time 60 (60 minutes incubation, black lines).

FIG. 2E is a graph showing a qPCR amplification curve (delta Rn) of RCA products prepared with the 60 minutes' pre-amplified samples of Exp. a, and Exp. b (shown in FIG. 2C and FIG. 2D, respectively). As a control, 108 synthetic dsDNA target was used in the ligation step without pre-amplification step (No RPA).

FIG. 3A is a gel analysis of RCA products for detection of mycoplasma pneumonia (Mp). Lane 1 was loaded with RCA products obtained from a sample containing 6.8×10² copies of the pathogen, Lane 2 was loaded with RCA products obtained from a sample containing 6.8×10¹ copies of the pathogen. Lane 3: Bordetella pertussis (Bp) gDNA 1×10⁴. Lane 4: Listeria monocytogenes (Lm) gDNA. Lane 5: double distilled water (DDW). Lane 6: synthetic Mp oligo. Lane 7: Uu (Ureaplasma urealyticum) oligo used as a positive control. The left lane is a Mw ladder.

FIG. 3B is a gel analysis of RCA products for detection of Chlamydia trachomatis (Ct). Lane 1 was loaded with RCA products obtained from a sample containing 10⁵ EBs of the pathogen, Lane 2 was loaded with RCA products obtained from a sample containing 10⁴ EBs of the pathogen. Lane 3: was loaded with RCA products obtained from a sample containing 10³ EBs of the pathogen. Lane 4: was loaded with RCA products obtained from a sample containing 10² EBs of the pathogen. Lane 5: was loaded with RCA products obtained from a sample containing 10 EBs of the pathogen. Lane 6: PBS pH=7.4. Lane 7: Vircell genomic DNA HSV 10² copies. Lane 8: synthetic DNA 0.1 nM CT T1. The left lane (M) is a Mw ladder.

FIG. 3C is a gel analysis of RCA products for detection of Neisseria gonorrhea (NG). Lane 1 was loaded with RCA products obtained from a sample containing 10⁷ cfu of the pathogen, Lane 2 was loaded with RCA products obtained from a sample containing 10⁶ cfu of the pathogen. Lane 3: was loaded with RCA products obtained from a sample containing 10⁵ cfu of the pathogen. Lane 4: was loaded with RCA products obtained from a sample containing 10⁴ cfu of the pathogen. Lane 5: was loaded with RCA products obtained from a sample containing 10³ cfu of the pathogen. Lane 6: PBS pH=7.4. Lane 7: Vircell genomic DNA NG 10² copies (T+RPA). Lane 8: synthetic DNA 0.1 nM NG T4. The left lane (M) is a Mw ladder.

FIG. 3D is a gel analysis of RCA products for detection of HSV1. Lane 1 was loaded with RCA products obtained from a sample containing 10⁷ pfu of the pathogen, Lane 2 was loaded with RCA products obtained from a sample containing 10⁶ pfu of the pathogen. Lane 3: was loaded with RCA products obtained from a sample containing 10⁵ pfu of the pathogen. Lane 4: was loaded with RCA products obtained from a sample containing 10⁴ pfu of the pathogen. Lane 5: was loaded with RCA products obtained from a sample containing 10³ pfu of the pathogen. Lane 6: PBS pH=7.4. Lane 7: Vircell genomic DNA NG 10² copies (T+RPA). Lane 8: synthetic DNA 0.1 nM NG T4 (T+RCA). The left lane (M) is a Mw ladder.

FIG. 4 is a gel analysis of RCA products for detection of SARS-Cov-2 in clinical samples. Lanes 1 to 4 were loaded with RCA products obtained from clinical samples. Lane 5: non-specific gDNA. Lane 6: synthetic SARS-Cov-2. Lane 7: synthetic UU oligo. Lane M: Mw ladder.

FIG. 5 shows RCA products of clinical samples detected using sensor imaging. The top panel shows the sensor design. The sensor comprises pathogen specific capture nucleic acids for: RSV B (respiratory Syncytial virus B), Flu B (influenza B), and CoV2 (SARS-COV-2). Each of the lower panels represents results obtained with a clinical sample containing the indicated pathogen. Highlighted spots represent a positive result. The dots on the right and left sides represent positive control for the hydrogel integrity.

FIG. 6 is a graphic representation of the method of the invention implemented on a cartridge.

FIG. 7 is a gel analysis of Gp32 HR products and Gp32 HR+RCA products for detection of Influenza A (FluA). Lane M: a Mw ladder. Lane 1: Gp32 HR products obtained from a sample containing 5,000 copies of the pathogen, Lane 2: Gp32 HR+RCA products obtained from a sample containing 5,000 copies of the pathogen. Lane 3: Gp32 HR products obtained from a sample containing 1,000 copies of the pathogen. Lane 4: Gp32 HR+RCA products obtained from a sample containing 1,000 copies of the pathogen. Lane 5: Gp32 HR products obtained from a sample containing 5,000 copies of the pathogen, Lane 6: Gp32 HR+RCA products obtained from a sample containing 5,000 copies of the pathogen. Lane 7: Gp32 HR products obtained from a sample containing 1,000 copies of the pathogen. Lane 8: Gp32 HR+RCA products obtained from a sample containing 1,000 copies of the pathogen. Lane 9: synthetic target FluA which underwent only RCA and served as the RCA control. Lane 10: double distilled water (DDW) (negative control).

FIG. 8 is a gel analysis of Gp32 HR+RCA products for detection of Influenza A (FluA). Lane M: a Mw ladder. Lanes 1 and 2: sample containing 1,000 copies of Chlamydophila Pneumonia. Lanes 3 and 4: sample containing 500 copies of FluA. Lanes 5 and 6: sample containing 100 copies of FluA. Lanes 7 and 8: sample containing 50 copies of FluA. Lane 9: synthetic target FluA (positive control). Lane 10: double distilled water (DDW) (negative control).

FIG. 9 is a gel analysis of Gp32 HR+RCA products and RCA only (no prior amplification with Gp32 HR) products for detection of Mycoplasma pneumoniae. Lane M: a Mw ladder. Lanes 1 and 2: Gp32 HR+RCA products obtained from a sample containing 100 copies of the pathogen. Lanes 3 and 4: RCA products obtained from a sample containing 100 copies of the pathogen. Lanes 5 and 6: Gp32 HR+RCA products obtained from a sample containing 1,000 copies of the pathogen. Lanes 7 and 8: RCA products obtained from a sample containing 1,000 copies of the pathogen. Lane 9: synthetic target MP which underwent only RCA and served as the RCA control. Lane 10: double distilled water (DDW) (negative control).

### DETAILED DESCRIPTION OF EMBODIMENTS

The present invention provides sensitive, specific, and multiplex identification/detection of target nucleic acid molecules in a sample. Specifically, the identification/detection of pathogens in clinical samples, with a significantly low limit of detection as compared to alternative methods.

The system and methods of the invention offer rapid, easy (sample-to-answer), accurate, and comprehensive molecular diagnosis of various infectious agents in near-patient settings. The methods of the invention can be performed using a fully automated system.

The detection method of the invention comprises two isothermal amplification steps:
(a) a first amplification step of converting a target nucleic acid (e.g., genomic DNA or RNA) into an amplicon, also referred to as the “preamplification step” and
(b) a second amplification step which employs rolling circle amplification (RCA) methodology performed using circular probes to further amplify a detection signal thereby achieving specific detection of low levels of the target nucleic acid.

The technology can be implemented in a variety of different formats, including microfluidic platforms and point-of care devices, for example on an electrophoretic chip, e.g., as described in WO 2018/122856 incorporated herein by reference.

The first “preamplification” (PA) step which converts the genomic DNA or RNA into an amplicon, provides an enhanced starting point for the RCA thus significantly increasing its sensitivity and the outcome of the specific multiplex pathogens diagnostics method. This step may be performed using any method known in the art for nucleic acid amplification. Two such non limiting methods are exemplified herein, namely recombinase polymerase amplification (RPA) and homologous recombination, particularly strand displacing DNA polymerase mediated homologous recombination, with or without Gp32.

As used herein the term “detection” or “detecting” refers to discovering or evidencing the presence of a target nucleic acid in a biological/clinical sample. In one specific aspect of the invention, the term detecting refers to examining whether one or more pathogens are present in the biological/clinical sample.

As used herein the term “pathogen” refers to an infectious agent that may be a bacterium or a virus.

According to an embodiment, the infectious agent is a virus, such as an RNA virus (single stranded or double stranded) or a DNA virus (single stranded or double stranded).

According to certain embodiments, the virus is an RNA virus, e.g. an RNA virus of the Coronaviridae Family, particularly a virus of the species severe acute respiratory syndrome related coronavirus, more particularly SARS-Cov-2.

According to certain embodiments, the virus is an RNA virus, e.g. an RNA virus of the Paramyxoviridae Family, particularly human respiratory virus (HSV) A or B.

According to certain embodiments, the virus is an RNA virus, e.g. an RNA virus of the Orthowoviridae Family, particularly influenza A or influenza B.

According to certain embodiments, the virus is a DNA virus, e.g. a DNA virus of the Herpesviridae family, particularly herpes simplex virus (HSV) 1 or 2.

According to an embodiment, the infectious agent is a bacterium. The bacterium is for example a bacterium responsible for respiratory infections such as Chlamydia pneumonia, Bordetella pertussis, Mycoplasma pneumonia, Mycobacterium tuberculosis, or Klebsiella pneumonia, or a bacterium responsible for sexually transmitted infections such as Chlamydia trachomatis, or Neisseria gonorrhea.

According to other embodiments, the bacterium is for instance E. Coli, Salmonella, Shigella, Pseudomonas aeruginosa, Borrelia burgdorferi, Vibrio Cholerae, Proteus mirabilis, or Enterococcus faecalis.

According to the method of detection, a single or multiplexed assay may be conducted to detect simultaneously one or more infectious agents.

The method generally comprises:
a. extraction of nucleic acids from a sample, comprising lysis and elution steps.

As used herein the term “sample” encompasses any sample that comprises biological material and may contain an infectious agent. It may be a clinical sample obtained from a human subject, or a biological sample obtained from an animal. The sample may be obtained from a bodily fluid or tissue such as blood, urine, feces, lung aspirate, cerebrospinal fluid, or semen. It also encompasses samples of water, soil or air, or samples that were taken from various surfaces that may contain biological material.

The lysis step is performed in a lysis solution. In one embodiment the lysis solution comprises Guanidinium Thiocyanate, sodium N-lauroylsarcosinate, isopropanol (IPA), carrier RNA and a protease. The lysis solution may further comprise Dithiothreitol (DTT), e.g., 80 mM DTT.

In a specific embodiment the lysis solution is as described in the Examples below, namely a solution comprising 50% Solution A (2.5M Guanidinium Thiocyanate and 0.7% sodium N-lauroylsarcosinate in 0.1M Tris-HCl pH 7.4), 35%-50% Isopropanol, carrier RNA (1 mg/ml), DDW, and a protease (e.g., Proteinase K). The extracted DNA is washed with a buffer, optionally comprising up to 70% ethanol. The wash may include one or more washing steps.

In an embodiment, the extracted DNA is subjected to two washing steps, the first with a buffer comprising 70% ethanol (e.g., Tris 66.8 nM, pH-7 KCl 330.3 mM+70% ethanol) and the second with a buffer comprising 30% ethanol (e.g., Tris 66.8 nM, pH-7 KCl 330.3 mM+30% ethanol).
b. Target specific amplicon generation.

Selective nucleic acid amplification is performed for example by isothermal amplification. The specificity is achieved by using target specific (e.g., pathogen specific) primer sequences. This step is also referred to as the “amplicon generation step” or “preamplification step”.

As used herein, the term “amplicon” refers to a piece of DNA or RNA that is the product of an amplification event Amplicons are nucleic acid sequences that are accumulated because of the amplification event. Thus, converting said target nucleic acid into an amplicon, means that the target nucleic acid undergoes an initial step of amplification, also referred to herein as preamplification.

In one embodiment, the isothermal amplification is performed using recombinase polymerase amplification (RPA) technique.

The isothermal amplification is optionally performed in the presence of a reverse transcriptase enzyme, such that if RNA viruses are present in the sample their genomic RNA is reverse transcribed into cDNA. For testing panels that are dedicated to indications in which RNA viruses are not likely to be involved (e.g., for testing patients with tuberculosis or sepsis), then the reverse transcriptase may be excluded from the reaction.

The RPA reaction disclosed herein contains a recombinase, which may originate from prokaryotic, viral or eukaryotic origin. Exemplary recombinases include RecA, and uvsX obtained from any species, e.g., the RecA/Rad51 ortholog of bacteriophage T4, and fragments or mutants thereof, and combinations thereof.

The RPA reaction disclosed herein contains a DNA polymerase, which may be any polymerase suitable for isothermal amplification, for example Klenow exo, Bsu large fragment, phi29, or the large fragment of Bst DNA polymerase.

In one embodiment, the nucleic acid elution step is combined with the RPA to reduce protocol steps, also referred to herein as “combined elution/RPA”. In such a case, the reaction may be performed using a dedicated recombinase reaction elution buffer comprising hydrochloride solution (pH 8.0), Potassium acetate, polyethylene glycol (PEG) or any other hydrophilic polymer, DTT (Dithiothreitol), dNTPs, ATP, Creatine phosphate, MgAc, and forward and reverse primers.

The RPA enzymes are added after the elution step is completed, and the reaction is allowed to proceed by providing suitable conditions (e.g., incubation in 400° C. for 5 to 20 minutes).

In another embodiment, the preamplification step is performed using homologous recombination. The method of the invention may be performed with any means for accomplishing homologous recombination. One non-limiting example to achieve homologous recombination is by using the enzyme Gp32 thus mimicking the bacteriophage T4 homologous recombination (HR) machinery. Alternatively, homologous recombination may be achieved by using a DNA polymerase and creating conditions to allow hybridization of a primer into the sequence of interest in the absence of Gp32. Then, for example with the help of “outer primers” (namely, primers that are located upstream to the forward preamplification primer and downstream to the reverse preamplification primer), the newly synthesized strand is displaced from the parental DNA and used as a template for an isothermal pre-amplification reaction.

Thus, in some embodiments, the homologous recombination is performed using a strand-displacing DNA polymerase with or without the presence of bacteriophage T4 Gp32.

According to one embodiment, the extracted nucleic acids are contacted with suitable primers, bacteriophage T4 Gp32, and a strand-displacing DNA polymerase. The reaction is performed in a buffer comprising a buffering agent (e.g., Tris-HCl pH 8.0, potassium acetate and MgAc (or alternatively, NaCl and MgCl), DTT or TECP (Tris(2-carboxyethyl) phosphine hydrochloride), and dNTPs.

According to another embodiment, the extracted nucleic acids are contacted with suitable primers, and a strand-displacing DNA polymerase. The reaction is performed in a buffer comprising a buffering agent, magnesium sulfate, and dNTPs. Advantageously the duration of the preamplification step (comprising a polymerization reaction and optionally reverse transcription) is very short, about 10 minutes, and it may even be shortened. It may be performed in a multiplex manner (i.e., 5 plex, 10 plex, or even 100 plex), and when combined with RCA (as will be described below) its limit of detection is lower than 50 copies (as shown for example for influenza A). It must be emphasized that this does not appear to be the lowest limit of detection, which is apparently lower than 50 copies, in the single digit range.

The homologous recombination preamplification step may be performed using any isothermal strand-displacing DNA polymerase. Non-limiting examples include Bsu, BST (e.g., BST3 large fragment), Phi29, and Klenow exo.

The homologous recombination preamplification step may be performed using two or more primers (namely, a primer set comprising at least a forward and a reverse primer) or using a single primer for each target. Use of a single primer may increase the extent of multiplexing, and it may also facilitate a quantitative reaction.

It is to be noted that for the detection of RNA viruses, such as, but not limited to, SARS Cov2 influenza or HSV, the extracted nucleic acids undergo reverse transcription prior to the amplification step.

The reaction may there fore be performed in the presence of a reverse transcriptase (RT) (e.g., RevertAid RT).

The use of the HR reaction enables an ethanol tolerance of up to about 30% in the preamplification step. This is a highly advantageous feature since ethanol wash improves the efficiency of nucleic acid purification. However, its use within a cartridge is generally avoided since many enzymatic reactions have a low ethanol tolerance and the ethanol cannot be removed by drying when used within a cartridge. Therefore, high tolerance of the HR reaction to residual ethanol facilitates the use of an ethanol washing step in a cartridge even though no drying of the ethanol can take place.
c. Open circular probe hybridization with the previously amplified target nucleic acid (the previously generated amplicon).

Short circular probes (also referred to as “padlock probes”) matching the target nucleic acid sequences at both the 3′ and 5′ ends were designed. A perfect match at the target region between the target nucleic acid and the padlock probe allows circularization of the padlock probe in the presence of thermostable ligase.

The hybridization may be performed in solution, in a semi solid interface, in a bead assay, or on a solid support.

In one embodiment, the hybridization is performed using a bead assay whereby the open circular probe is attached to a magnetic bead. The attachment may be performed for example via biotin-streptavidin binding, namely streptavidin magnetic beads are associated with the open circular probe via a biotinylated capture probe which has a sequence that is complementary to a sequence within the open circular probe.
d. Ligation of positive circular probes which showed specific recognition.

After the circular probe hybridizes with its target sequence the probe is ligated using a suitable enzyme. Non-limiting examples of ligation enzymes include Hifi Taq ligase, ligase, taq ligase, 9°N DNA, ligase t4, and splintR ligase.

e. Elimination of non-ligated probes and nucleic acids. For example by exonuclease treatment (e.g., using exonuclease I).

f. Rolling circle amplification (RCA) of positive probes for signal enhancement. The circularized padlock probe is then amplified with a DNA polymerase (e.g., BST, Klenow exo, Phi29, Bsu or Vent exo-DNA polymerase which are active in a temperature range of 37° C. to 70° C.) using primers derived from the central region (backbone) of the circular probe, in the presence of nucleotides. The reaction is performed with forward and reverse branching primers to exponentially amplify the produced signal, also referred to as hyper-branched RCA. In linear RCA, only a forward primer is used, and therefore a linear, long DNA strand is produced. In hyper-branched RCA, a reverse primer is included (the forward and reverse primers are also referred to as sense and antisense primers) to increase the amount of synthesized DNA and decrease the incubation time. As a result, a reverse strand is produced, which, in turn, becomes a template for the forward primer. This process is called branching.

Thereby along concatemeric single stranded DNA (ssDNA) is produced with tens to hundreds of tandem repeats.

The RCA is conducted at a constant temperature, e.g., 65° C. as shown in the Examples below.

The RCA may be performed in the presence of the enzyme nickase which nicks only one strand of the nucleic acid and thereby generates additional priming sites for the polymerase amplification action which creates branching, thereby further amplifying the signal. The suitable nickase is selected according to the unique target sequences, e.g., BSTNBI nicking endonuclease.
g. Cutting RCA products to short, oligos, also referred to herein as “concatemer digestion”.

The suitable cutting enzymes are selected according to the unique target sequences. Non-limiting examples for enzymes that can be used for cutting the RCA products include Haelll, Nhelll, and AluI.
h. Detection.

Amplified DNA can be detected using any method known in the art. Nonlimiting example include hybridization and detection of amplified targets on screen printed carbon electrodes with fluorescent probes followed by sensor imaging, UV illumination after staining with Cybr-gold® (Molecular Probes), and molecular beacons. Compatible buffers are used for these reactions

As used herein, the term “fluorescent probe” refers to any substance that emits electromagnetic energy at a certain wavelength (emission wavelength) when the substance is illuminated by radiation of a different wavelength (excitation wavelength) and is intended to encompass a chemical or biochemical molecule or fragment thereof that is capable of interacting or reacting specifically with an analyte of interest in a sample to provide one or more optical signals.

Representative fluorescent probes for use in the methods provided herein include for example, Cy5, Cy3, fluorescein amidites (FAM), and the like.

An embodiment of sensor imaging is demonstrated in the Examples below. Accordingly, the RCA products that were obtained by the amplification process of the invention were digested and electronically addressed to active sites/electrodes. Namely, since the digested RCA products or amplicons are negatively charged, they are electrically addressed (pulled) into a hydrogel, where capture-molecules bind complementary amplicons. In one embodiment, a power flow (current) of 200 μAmp is applied for 30 seconds to perform the addressing.

As used herein the term “addressing” refers to the electrical pulling of the RCA products or amplicons into a hydrogel. In an embodiment, the addressing is performed on an electrophoretic chip, for example as described in WO 2018/122856 incorporated herein by reference.

Next, the fluorescent report complex, comprising the reporter oligo with 3′ and 5′ fluorophores (which are selected depending on the filters in the imaging system, for example but not limited to Cy5), which binds to the complementary sequence on the circular probe (CP) and to an additional oligo (e.g., 60 bp long) which complementarily binds the CP and additional reporter molecules (e.g., two or more). Resulting in multiple fluorophore molecules signaling for each CP captured on the array. The reporter fluorochrome is excited at the appropriate wavelength (e.g., 630 nm), and the sensor imaging system detects the emitted light (e.g., 670 nm) in an optical test chamber. In one embodiment, the detection process is carried out in a single tube, microplate well, liquid handler or cartridge. For example, the cartridge and systems for computerized automatic diagnosis disclosed in WO 2019/130,309, WO 2020/008463, WO 2018/122852, WO 2019/130290., incorporated herein by reference.

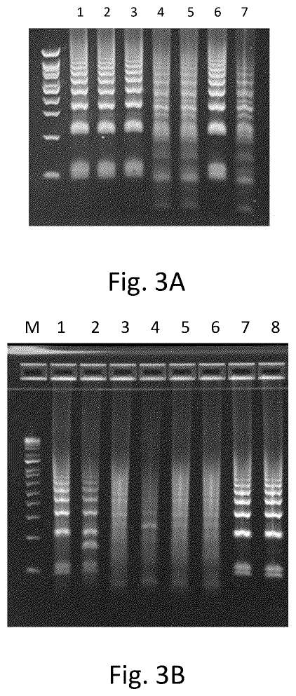

When the detection is performed in a cartridge the movement between the compartments is based on microfluidics. The invention thus encompasses assays integrated on microfluidics and one-step detection assays, in which a sample is introduced onto a cartridge and an assay report is produced showing the results of the multiplex detection (as exemplified in FIG. 6).

In a specific embodiment, the method provides primers and probes for detection of indication-specific pathogens, also referred to herein as “indication-specific panels”, or “panels”. The panels may include, but are not limited to, pathogens affecting the respiratory system, CNS-associated infections, sexually transmitted diseases, gastrointestinal diseases, drug resistant pathogens, plant, and animal pathogens (particularly plants/animals of agricultural relevance or pets), and sepsis.

As used herein the term “sexually transmitted diseases” (also referred to as sexually transmitted infections, or STI) includes, but, is not limited to Chlamydia trachomatis, Neisseria gonorrhea, and HSV1, as exemplified below.

As shown in the Examples below, a panel of 8 respiratory pathogens (4 viruses and 4 bacteria) was developed and verified for LOD and clinical parameters from clinical samples for sensitivity and specificity, both over 99%. The method of the invention therefore provides a low limit of detection (LOD) of 10 to 100 copies in a reaction. Furthermore, the novel method of the invention allows the inclusion of this process in a single cartridge for sample-to-answer in vitro diagnostics.

In one of its aspects, the invention provides a system featuring a compact, easy-to-use, benchtop analyzer utilizing unique multiple sensors and a disposable fully integrated cartridge allowing the fully automatic sample-to-answer processing of clinical samples in 30-45 min. It features a high multiplexing capacity (up to 100 molecular targets per sample). The method of the invention is carried out within the cartridge. The system can be designed for any application or symptomatic condition and can be deployed as a point of care for a pandemic/endemic solution.

### EXAMPLES

According to the invention, standard methods may be used for preparing recombinant nucleic acids, culturing cells, and introducing nucleic acids, in particular RNA into cells, specifically lipofection, for example as described below.

### Materials and Methods

### Nucleic Acid Extraction, Reverse Transcription, and Pre-Amplification

Lysis and extraction: 600 μl lysis solution (50% Solution A (2.5M Guanidinium Thiocyanate and 0.7% sodium N-lauroylsarcosinate in 0.1M Tris-HCl pH 6.5), 35% Isopropanol, 6 μl carrier RNA (1 mg/ml), 20 μl Speed Beads Magnetic Carboxylate, and DDW), and 40 μl Proteinase K were added to 200 μl of the clinical sample and vortexed for 30 sec. The mixture was then incubated for 2 minutes at 80° C. The tube was then transferred into a magnet holder and magnetized for 20 seconds until the beads were collected on the wall of the tube. Supernatant was discarded and the tube was removed from the magnet.

The extracted DNA was washed with a Wash buffer I: Tris 66.8 nM, pH-7 KCl 330.3 mM+70% ethanol, followed by a second wash with a Wash buffer II: Tris 66.8 nM, pH-7 KCl 330.3 mM+30% ethanol.

Nucleic acid elution in recombinase reaction buffer: The nucleic acids were eluted by adding 55 μl elution buffer. The elution buffer contained: 32.4 μl recombinase reaction buffer (RRB): 50 mM Trizma hydrochloride solution (pH 8.0), 160 mM Potassium acetate, 5% PEG (20000), 2 mM DTT, 200 μM dNTPs, 3 mM ATP, 50 mM Creatine phosphate, 14 mM MgAc, 9.2 μl of a 10 μM primer stock solution of each forward and reverse primer listed in Table 1, 2.75 μl magnesium acetate and 10.6 DDW. The beads were resuspended in the elution buffer and incubated for 2 minutes at 56° C. The tube was spun briefly, placed on a magnet holder, and magnetized for 20 seconds until the beads were collected on the wall of the tube. The eluted nucleic acids were collected.

TABLE 1
RPA primer sequences

| | | | Amplicon | SEQ |
|---|---|---|---|---|
| | Pathogen | Sequence | length | ID |
| Name | (Gene target) | (5′-3′) | (bp) | NO |
| Fr_Chp | Chlamydia | GTA AAG AAT AAC | 127 | 1 |
| | pneumonia | GAG CAC CAC GCT | | |
| | (Arginine | CTC CAG CAA CC | | |
| Rv_Chp | receptor) | CAG GAG GAA TTA | | 2 |
| | | TGT GCA AAA CTC | | |
| | | TTA GCT CAA GG | | |
| Fr_LP | Legionella | ATT GTT TAT GAA | 123 | 3 |
| | pneumophila | GAT GAA ATT GGT | | |
| | (mip) | GAC TGC GGC TG | | |
| Rv_LP | | CCA ATG CTA TAA | | 4 |
| | | GAC AAC TTA TCC | | |
| | | TTG TCT GTA GC | | |
| Fr_Mp | Mycoplasma | CAT TGG CTT GGG | 146 | 5 |
| | pneumonia | TTT GCA GCT CTT | | |
| | (ADP- | GAT AAT GAG GG | | |
| Rv_Mp | ribosylating | TGT CCG TAG TGC | | 6 |
| | toxin CARDS) | TTG ACT AGT AGA | | |
| | | TGC TGT TCC CG | | |
| Fr RSVA | Respiratory | TTA TGA ATT ATA | 118 | 7 |
| | Syncytial Virus | CAC TCA ACA ATG | | |
| | Infection A | CC | | |
| Rv RSVA | (Fusion protein, | GCA ATG CCA CTG | | 8 |
| | F) | GCG ATT G | | |
| Rv RSVB | Respiratory | CCT CTT CTT GCT | 107 | 9 |
| | Syncytial Virus | TAT TGA TAC ATT | | |
| | Infection B | TAG G | | |
| Fr RSVB | (Fusion protein, | CAA GAA TTA GAT | | 10 |
| | F) | AAG TAT AAG AAT | | |
| | | GCA GTA ACA G | | |
| Fr FluA | Influenza A | GTT CTT TCT ATC | 152 | 11 |
| | (Segment 7, | ATC CCG TCA GGC | | |
| | matrix protein | CCC CTC AAA GC | | |
| Rv FluA | M1) | CCT AAA ATT CCC | | 12 |
| | | TTA GTC AGA GGT | | |
| | | GAC AAG ATT GG | | |
| FR FluB | Influenza B | GAG ACA TCA AAG | 181 | 13 |
| | (Segment 5, | CCA AGA CTG CCT | | |
| | nucleoprotein | ATG AAA AGA TTC | | |
| Rv FLuB | (NP) gene) | GCC ACA GAG GGC | | 14 |
| | | CTA ACA ACG ACC | | |
| | | ATA CTA CGA GC | | |
| Fr-Bp | Bordetella | TGG CGC GAG TCT | 114 | 15 |
| | pertussis | GCT GGC TGA CGT | | |
| | (ptxA) | AGC GAG CGT TG | | |
| Rv-Bp | | TTC CGC CCG AAA | | 16 |
| | | ACA TCC GCA GGG | | |
| | | TAA CGC GGG TC | | |
| Fr Covid19 | SARS-Cov-2 | ACA ATG TAA CAC | 114 | 17 |
| | (Protein N) | AAG CTT TCG GCA | | |
| | | GAC | | |
| Rv Covid19 | | GTG CAA TTT GCG | | 18 |
| | | GCC AAT GTT TGT | | |
| | | AAT C | | |
| RPA_CT_pbpB_ | Chlamydia | GGC AAA CAC TAA | 169 | 47 |
| T1_T4F | trachomatis | TAC CGA TTT CAA | | |
| | (PBP2- | G | | |
| RPA_CT_pbpB_ | transglycolase/ | GCT TTT ACT TGC | | 48 |
| T1_T4R | transpeptidase) | AAG ACA TTC CTC | | |
| RPA_NG_orf_ | Neisseria | CAG AAT TTG GCG | 163 | 49 |
| T1_T4F | gonorrhea | TAC CTC AAT TTC | | |
| | (orf1) | G | | |
| RPA_NG_orf_ | | TGT GGA ATA TTG | | 50 |
| T1_T4R | | GAT ATG GCC TGA | | |
| | | C | | |
| RPA_HSV-1 | Herpes Simplex | CCA CAG GGT CAG | 134 | 51 |
| Forward | Virus 1 (HSV1) | CTC GTG ATT CTG | | |
| | (glycoprotein | C | | |
| RPA_HSV-1 | B) | ATC AAG ACC ACC | | 52 |
| Reverse | | TCC TCC ATC GAG | | |

cDNA synthesis and pre-amplification: The eluent was added to a mix of uvsY (a bacteriophage T4 recombination mediator protein (RMP) which promotes homologous recombination), uvsX recombinase (the RecA/Rad51 ortholog of bacteriophage T4), gp32 (The Gp32 protein of bacteriophage T4 plays critical roles in DNA replication, recombination, and repair by coordinating protein components of the replication fork and by stabilizing ssDNA), Bsu (a DNA polymerase), Phospho Creatine Kinase (optional), and RevertAid Reverse Transcriptase, mixed gently, and incubated for 20 min at 40° C. The RNA of RNA-based pathogens was thus reverse transcribed into cDNA to allow formation of the amplicon.

This step results in the production of amplicons of the detected pathogens. These amplicons are next subjected to further amplification using a rolling circle amplification method.

### Binding and Ligation

### Ligation Beads Preparation:

30 μl of Streptavidin Magnetic Beads were aliquoted into a RNase-free microcentrifuge 1.5 ml tube. A magnet was applied to the side of the tube for 20 seconds and the supernatant was discarded. 30 μl of Wash Buffer 1 (1M NaCl, 40 mM Tris-HCl pH 7.5) were added to the tube and pipetted to suspend beads. A magnet was applied once again to the side of the tube for 20 seconds and the supernatant was discarded.

Cp+F primer (circular probe+forward primer) mix (Volume per sample: 3 μl of the 5′ biotinylated forward capture primer 5Bio_Fr_BSTNBI which is rich in TC repeats (at a final concentration of 1 μM in binding buffer (0.5M NaCl, 40 mM Tris-HCl pH 7.5), 0.6 μl per circular probe of CP_XXX_BSTNBI_HaeIII_TN (final concentration 0.02 μM in binding buffer), and 26.4 μl binding buffer) were added to the beads, mixed, and incubated for 5 minutes at room temperature. The primer and the circular probe bind to each other through complementary sequences.

The 5′-3′ sequence of 5Bio_Fr_BSTNBI is TCT CTC TCT CTC TCT CTC TCT CTC GAG TCG CTA CCT TGC CCT AAA CG (SEQ ID NO. 19). 5-Bio primer for CP+F mix preparation+TC repeats.

A list of the circular probes is provided in Table 2.

TABLE 2
Sequences of the circular probes

| | | | Sequence | |
|---|---|---|---|---|
| | | Sequence | length | SEQ |
| Name | Pathogen | (5′-3′) | (bp) | ID NO |
| CP | Bordetella | TCG CCG GTG ATG CCG | 117 | 20 |
| Bp_BSTNBI_ | pertussis | TTG TGA GGC GTT TAG | | |
| HaeIII_TN | | GGC AAG GTA GCG ACT | | |
| | | CTT TTA GCC GTT CCT | | |
| | | CGG CCC AGA CTT TTC | | |
| | | AAC AGA CAG GAA CAG | | |
| | | GCT TTT GAA TAC TCC | | |
| | | GTG GTC GTG GTC | | |
| CP_Covid19_ | SARS-Cov2 | TGG ACT TTA TTG ACA | 123 | 21 |
| ORFab2_ | | CTA AGA GGG GAG GCG | | |
| anti_BSTNBI_ | | TTT AGG GCA AGG TAG | | |
| TN_HaeIII | | CGA CTC TTT TAG CCG | | |
| | | TTC CTC GGC CCA GAC | | |
| | | TTT TCA ACA GAC AGG | | |
| | | AAC AGG CTT TTT CAT | | |
| | | GCA CTT TGT CCG AAC | | |
| | | AAC | | |
| CP_FLUA#1_ | Influenza A | GTC TTT GCA GGA AAG | 120 | 22 |
| anti_BSTNBI_ | | AAC ACA GAT AGG CGT | | |
| TN_HaeIII_ | | TTA GGG CAA GGT AGC | | |
| 44 bp | | GAC TCT TTT AGC CGT | | |
| | | TCC TCG GCC CAG ACT | | |
| | | TTT CAA CAG ACA GGA | | |
| | | ACA GGC TTT TTC GCG | | |
| | | CAG AGA CTG GAA AGT | | |
| CP_ | Influenza B | AGT TGA TCA AGT GAT | 116 | 23 |
| influenzaB_ | | CGG AAA GGC GTT TAG | | |
| anti_TN_ | | GGC AAG GTA GCG ACT | | |
| long BstNBI | | CTT TTA GCC GTT CCT | | |
| | | CGG CCC AGA CTT TTC | | |
| | | AAC AGA CAG GAA CAG | | |
| | | GCT TTT GCG CCC CAA | | |
| | | CAA AAG GCT CT | | |
| CP RSV A | Respiratory | TCT TGG TTT TTT GTT | 111 | 24 |
| gene F_ | Syncytial | AGG AGG CGT TTA GGG | | |
| anti_BstNBI_ | Virus | CAA GGT AGC GAC TCT | | |
| TN_HaeIII | Infection A | TTT AGC CGT TCC TCG | | |
| | | GCC CAG ACT TTT CAA | | |
| | | CAG ACA GGA ACA GGC | | |
| | | TTT TAA AAG GAA AAG | | |
| | | AAG ATT | | |
| CP RSV B | Respiratory | ACC ACA GTA TAT GAA | 118 | 25 |
| Antisense_ | Syncytial | CTA CAC AGG CGT TTA | | |
| BstNBI_TN | Virus | GGG CAA GGT AGC GAC | | |
| | Infection B | TCT TTT AGC CGT TCC | | |
| | | TCG GCC CAG ACT TTT | | |
| | | CAA CAG ACA GGA ACA | | |
| | | GGC TTT TCA ACC GGG | | |
| | | CTA GAA GAG AAG C | | |
| CPnew2_ | Mycoplasma | CAT GAC CAG GTT CAA | 115 | 26 |
| Mp_BstNBI_ | pneumonia | CGG GAA CAG GCG TTT | | |
| HaeIII_TN | | AGG GCA AGG TAG CGA | | |
| | | CTC TTT TAG CCG TTC | | |
| | | CTC GGC CCA GAC TTT | | |
| | | TCA ACA GAC AGG AAC | | |
| | | AGG CTT TTA CCA GCC | | |
| | | GGG TGG TGA G | | |
| CP_ChP_ | Chlamydia | AAC GAG ATA CAG ACG | 118 | 27 |
| BstNBI_ | pneumonia | ACT GGG AGG CGT TTA | | |
| HaeIII_TN | | GGG CAA GGT AGC GAC | | |
| | | TCT TTT AGC CGT TCC | | |
| | | TCG GCC CAG ACT TTT | | |
| | | CAA CAG ACA GGA ACA | | |
| | | GGC TTT TCA GCC TGA | | |
| | | ATC TTT CGT AGC C | | |
| CP_Lp_ | Legionella | CAT TGC AAG CCC CAT | 118 | 28 |
| BstNBI_ | pneumonia | AAC AGC AGG CGT TTA | | |
| HaeIII_TN | | GGG CAA GGT AGC GAC | | |
| | | TCT TTT AGC CGT TCC | | |
| | | TCG GCC CAG ACT TTT | | |
| | | CAA CAG ACA GGA ACA | | |
| | | GGC TTT TGG TTG CAG | | |
| | | CCA TTG CTG TTG A | | |
| CT_pbpT1 | Chlamydia | CTA GGA CGC AGT GCC | 116 | 53 |
| | trachomatis | GCC AGA GGC GTT TAG | | |
| | | GGC AAG GTA GCG ACT | | |
| | | CTT TTA GCC GTT CCT | | |
| | | CGG CCC AGA CTT TTC | | |
| | | AAC AGA CAG GAA CAG | | |
| | | GCT TTT GAC ATC TTG | | |
| | | TCT GGC TTT AA | | |
| NG_orf_T4 | Neisseria | CGG AAC CGA CGC ACA | 112 | 54 |
| | Gonorrhea | ATG AGG CGT TTA GGG | | |
| | | CAA GGT AGC GAC TCT | | |
| | | TTT AGC CGT TCC TCG | | |
| | | GCC CAG ACT TTT CAA | | |
| | | CAG ACA GGA ACA GGC | | |
| | | TTT TGA TTT GAT TTT | | |
| | | CGC CAT C | | |
| HSV-1_gb_T2 | HSV1 | CAT GTC AAC GAT ATG | 116 | 55 |
| | | TTG GGA GGC GTT TAG | | |
| | | GGC AAG GTA GCG ACT | | |
| | | CTT TTA GCC GTT CCT | | |
| | | CGG CCC AGA CTT TTC | | |
| | | AAC AGA CAG GAA CAG | | |
| | | GCT TTT CGT ACA ACC | | |
| | | ACA TAC AGC GC | | |

A magnet was then applied to the side of the tube for 20 seconds and the supernatant was discarded. The beads were then washed twice with the Binding Buffer followed by washing with Wash buffer 2 (150 mM NaCl, 40 mM Tris-HCl pH 7.5), separated using a magnet and the supernatant was discarded. 5 μl ligation mix (0.11M DL-Dithiothreitol solution, 11 nM NAD⁺, Hifi Taq ligase X11, in ultrapure DNase/RNase free water) were added to the beads and pipetted to resuspend the beads in the solution.

### Binding and Ligation Procedure:

Denaturation step: The eluent obtained after the preamplification step described above was incubated for 2 minutes at 95° C. Aliquots (5 μl) of beads (associated with the circular probe) in ligation mixture containing, 11 nM nicotinamide adenine dinucleotide (NAD+), and Hifi Taq ligase X11, were added to 50 μl of the denatured eluent and incubated for 10 minutes at 65° C.

Exonuclease treatment: At the end of the ligation, 2 μL of Exonuclease I were immediately added to the reaction (for a final concentration of 0.7 units/μl), mixed and incubated for 5 minutes at 65° C.

### Amplification (RCA)

At the end of the Exonuclease treatment step, the beads were separated using a magnet and the supernatant was discarded. 30 μl RCA mix was added to the beads. The RCA mix was prepared as follows: isothermal buffer concentration X1, dNTPs 1.4 mM, MgSO4 6 mM, Fr (forward)-branching primer (3′PTO) 2 μM, Rv (Reverse)-branching primer (3′PTO) 0.5 μM, second reverse primer RV-TN (3′PTO) 0.5 μM, BST polymerase 400 units/ml (total of 12 units/sample) in ultrapure Dnase/Rnase free water. The beads were suspended in the RCA mix and incubated for 15 minutes at 65° C.

### Example 1: Visualizing the RCA Products Using Sensor Imaging

In this example the final stage of the protocol was tested, namely the digestion of the RCA product and its addressing/reporting on an array.

The negatively charged DNA molecules (namely, the digested/cut RCA products or amplicons) are electronically addressed (pulled) into a hydrogel, where capture molecules bind complementary RCA products or amplicons. The power flow (current) of 200 μAmp is applied for 30 seconds to perform the addressing.

Separate samples each containing 0.5 nM of 120 bp single-stranded DNA that was used to begin the ligation step of Legionella pneumonia (Lp), Bordetella pertussis (Bp), Mycoplasma pneumonia (Mp) or Chlamydia pneumonia were subjected to the amplification process of the invention as described above. The RCA products that were obtained by the amplification process were digested and electronically addressed (200 μAmp/spot, 30 sec) to the active sites/electrodes.

Then the fluorescent reporter complex, comprising the reporter oligo with 3′ and 5′ Cy5 fluorophores which binds to the complementary sequence on the circular probe (CP) and to an additional 60 bp long oligo which complementary binds the CP and two reported molecules is added. Therefore, six fluorophore molecules signal for each CP captured on the array.

The reporter fluorochrome was excited at 630 nm, and the sensor imaging system caught the emitted light of 670 nm in an optical test chamber.

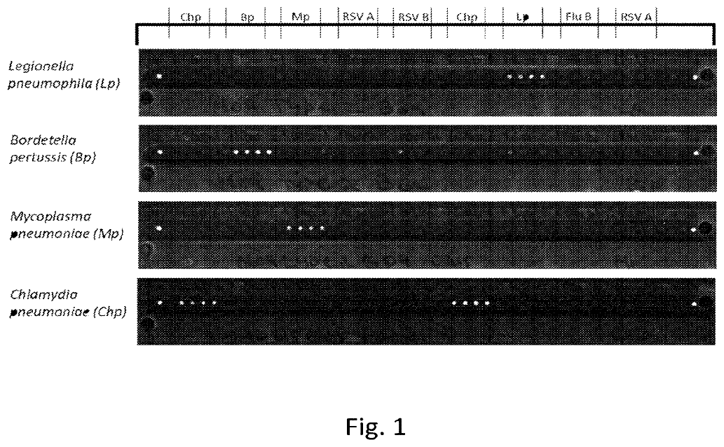

As can be seen in FIG. 1, the amplification process of the synthetic targets was sensitive and repetitive (as can be seen for example in the repetitive results obtained with Chlamydia pneumonia) resulting in a clear identification of the target pathogen with a high signal to noise ratio (as can be seen for example with the clear negative results obtained for RSV A, RSV B, and influenza).

### Example 2: Limit of Detection for Various Pathogens

To establish the limit of detection of the amplification method of the invention, samples containing various amounts of the target pathogen were used.

Bordetella pertussis

The samples contained 1000, 100, or 10 copies of the pathogen gDNA by Vircell. The RCA product was prepared as described in Example 1, using Bordetella pertussis specific primers as shown in Table 1 (SEQ ID Nos 15 and 16), and a circular probe as shown in Table 2 (SEQ ID No. 20).

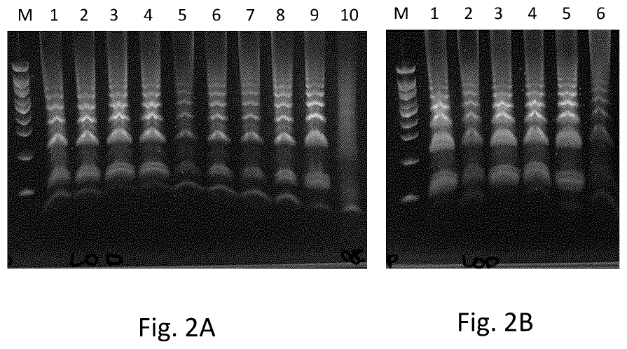

The RCA products were visualized on a gel (E-Gel® 2% Agarose, Thermo Fisher Scientific). As can be seen in FIGS. 2A and 2B, the method allowed the detection of as low as 10 copies of the pathogen per sample. Non-specific synthetic DNA, namely an out of panel synthetic target, served as a negative control. An RCA reaction produces DNA products containing multiplications of circular probes; hence the products appear as a ladder of bands on the agarose gel.

To account for potential background non-relevant signal, the Ct (cycle threshold) and the melting temperature (TM) values were measured for the RCA products.

Ct indicates the number of amplicons received after the RCA reaction. The Ct is defined as the number of cycles required for the fluorescent signal to exceed background levels. Ct levels are inversely proportional to the amount of target nucleic acid in the sample (i.e., the lower the Ct level, the greater the amount of target nucleic acid in the sample). Ct was measured according to a threshold value of 1. This value was chosen as it falls in the linear stage of the amplification curve.

Tm values are determined automatically by the QuantStudio Design (Applied Biosystmes by Thermo Fisher Scientific) and its Analysis software according to the peak in the melting curve.

Table 3 shows the Ct and TM values for the Bordetella pertussis RCA products.

TABLE 3
Ct and TM values

| Copies per | | |
|---|---|---|
| Sample | Ct | Tm |
| 10³ | 11.99 | 85.34 |
| 10³ | 11.55 | 85.19 |
| 10³ | 11.62 | 85.05 |
| 10³ | 11.86 | 85.05 |
| 10² | 14.35 | 84.90 |
| 10² | 12.19 | 84.90 |
| 10² | 12.58 | 84.75 |
| 10² | 12.24 | 84.90 |
| 10 | 11.68 | 85.20 |
| 10 | 12.67 | 85.02 |
| 10 | 11.14 | 85.17 |
| 10 | 11.20 | 85.05 |
| +DNA | 11.87 | 85.02 |
| nonspecific DNA | — | unspecific Tm |

Both the Ct and the TM values indicate that the signal is specific.

Next, the detection efficacy of the method of the invention which combines recombinase-based amplification (as a preamplification step) with RCA was compared to the detection efficacy of RCA alone. To address the exact amount of amplified product produced, double strain synthetic oligonucleotide (Bordetella pertussis) with a well-known number of copies was used as a template and DDW as negative control.

Reactions were performed at 40° C. for 30 min or 60 min. Standard conditions were 50 mM Tris (Sigma, T2694, pH 8.0), 160 mM potassium acetate (Sigma, 95843F), 28 mM magnesium acetate (Sigma, 63052), 2 mM DTT (Sigma, 1719757), 5% PEG 20.000 (Sigma 8.18897), 1.4 mM dNTPs (New England Biolabs: NEB, N0447L), 3 mM ATP (New England Biolabs: NEB, P0756S), 50 mM phosphocreatine (Sigma 19333-65-4), 100 ng/μL creatine kinase (Roche, 10127566001), and 0.33 U/L Bsu large fragment (New England Biolabs: NEB, M0330L). Recombinase protein concentrations were 700 ng/μL gp32 (New England Biolabs: NEB, M0300S), 200 ng/μL uxsX (Intact Genomics 3562), and 100 ng/μL uvsY (Intact Genomics 3572). Primers were used at concentration of 480 nM each. Reaction final volumes were 50 μL.

The ligation and the RCA steps were performed as described above.

FIGS. 2C and 2D show a qPCR amplification curve of the recombinase polymerase pre-amplification (RPA) reaction using specific primers to detect Bordetella pertussis amplicon. Namely the experiments were performed without a further amplification step (no RCA). The RPA reactions were done in duplicate, referred to as Exp. 1a (FIG. 2C) and Exp. 1b (FIG. 2D), in a single-plex mode using 108 synthetic dsDNA target. Three time points were analyzed: no incubation, 30 minutes incubation, and 60 minutes incubation. The reactions were diluted 1:100 in DDW prior to qPCR. A threshold value of 1 was used to calculate the Ct values. In Exp. 1a the Ct value at time 0 was 27.9, and it was reduced to 7.54 after 30 minutes incubation and to 6.85 after 60 minutes incubation. Similar results were obtained in Exp1b in which the Ct value at time 0 was 26.33, and it was reduced to 6.81 after 30 minutes incubation and to 6.58 after 60 minutes incubation. These values reflect a number of amplified copies of 5.96E+10 and 9.36E+10 after 30 and 60 minutes respectively in Expla, and a number of amplified copies of 9.50E+10 and 1.10E+11 after 30 and 60 minutes respectively in Explb.

FIG. 2E shows qPCR amplification curves of the RCA product amount. The RPA reactions after a 60-minute incubation (samples were taken from the experiments shown in FIGS. 2C and 2D) were used in the full RCA protocol. Namely the results present an amplification obtained after performing RPA+RCA. As a control, 108 synthetic dsDNA target was used in the ligation step without pre-amplification step.

A reduction of about 6-7 Ct values was observed in samples that underwent RPA+RCA as compared to samples without RPA, indicating that the combined amplification reactions result in a higher amount of amplified product.

Mycoplasma pneumonia (Mp)

The samples contained 680, or 68 copies of Mp gDNA by VirCell (a commercially available source of bacteria and viruses providing the number of copies per microliter, VirCell Microbiologists). The RCA product was prepared as described in Example 1, using Mycoplasma pneumonia specific primers as shown in Table 1 (SEQ ID Nos 5 and 6), and a circular probe as shown in Table 2 (SEQ ID No. 26).

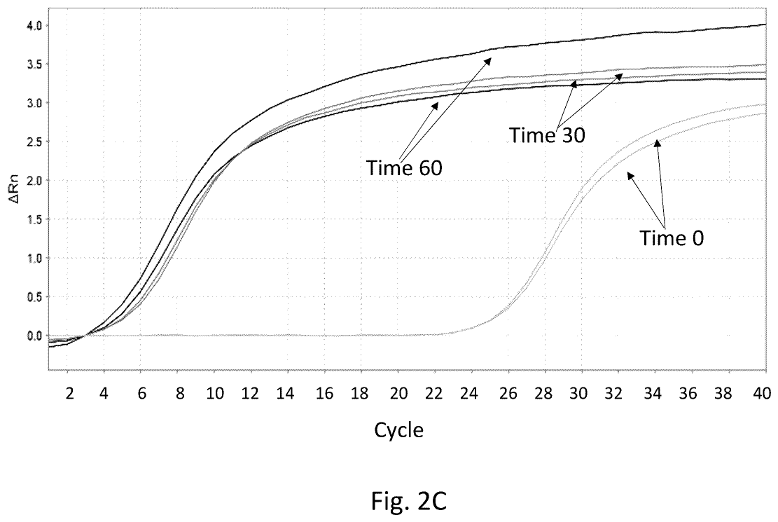

The RCA products were visualized on a gel. As can be seen in FIG. 3A, the method allowed the detection of as low as about 60 copies of the pathogen per sample. To account for potential background non-relevant signal, the Ct, and the melting temperature (TM) values were measured for the RCA products.

Table 4 shows the Ct and TM values for the Mycoplasma pneumonia RCA products, as compared with various controls.

TABLE 4
Ct and TM values

| | Copies per | | |
|---|---|---|---|
| Sample | Sample | Ct | Tm |
| M. pneumoniae | 6 × 10² | 7.65 | 85.04 |
| M. pneumoniae | 6 × 10¹ | 7.32 | 85.04 |
| Bordetella pertussis | 10⁴ | 7.52 | 86 |
| Listeria monocytogenes | | — | unspecific |
| | | | Tm |
| DDW | | — | unspecific |
| | | | Tm |
| M. pneumoniae | 5 nM | 7.73 | 85.04 |
| synthetic target | | | |
| Non-specific | 5 nM | — | unspecific |
| synthetic target | | | Tm |

Both the Ct and the TM values indicate that the signal is specific.

The limit of detection was also measured for Chlamydophila pneumonia, influenza B and SARS-Cov-2. For all these pathogens the limit of detection was in the range of 10 to 100 copies in a sample.

The method of the invention was also employed for the detection of several exemplary sexually transmitted infections (STI), namely Chlamydia trachomatis, Neisseria gonorrhea, and HSV1.

Chlamydia trachomatis (Ct)

The samples contained 10 elementary bodies (EBs), 10² EBs, 10³ EBs, 10⁴ EBs or 10⁵ EBs of Chlamydia trachomatis diluted in PBS. The RCA product was prepared as described in Example 1, using Chlamydia trachomatis specific primers as shown in Table 1 (SEQ ID Nos 47 and 48), and a circular probe as shown in Table 2 (SEQ ID No. 53). The RCA products were visualized on a gel. As can be seen in FIG. 3B, the method allowed the detection of as low as about 10⁴ EBs of the pathogen per sample.

Neisseria gonorrhoeae (NG)

The samples contained 10³ colony forming units (cfu), 10⁴ cfu, 10⁵ cfu, 10⁶ cfu, or 10⁷ cfu of Neisseria gonorrhoeae diluted in PBS. The RCA product was prepared as described in Example 1, using Neisseria gonorrhoeae specific primers as shown in Table 1 (SEQ ID Nos 49 and 50), and a circular probe as shown in Table 2 (SEQ ID No. 54). The RCA products were visualized on a gel. As can be seen in FIG. 3C, the method allowed the detection of as low as about 10⁵ cfu of the pathogen per sample.

### Herpes Simplex Virus-1 (HSV1)

The samples contained 10³ plaque forming units (pfu), 10⁴ pfu, 10⁵ pfu, 10⁶ pfu, or 10⁷ pfu of HSV1 diluted in PBS. The RCA product was prepared as described in Example 1, using HSV1 specific primers as shown in Table 1 (SEQ ID Nos 51 and 52), and a circular probe as shown in Table 2 (SEQ ID No. 55).

The RCA products were visualized on a gel. As can be seen in FIG. 3D, the method allowed the detection of as low as about 10⁵ pfu of the pathogen per sample.

Table 5 shows a non-limiting example of the extraction yield by quantifying HSV1 DNA immediately after extraction.

TABLE 5
extraction yield

| | HSV1 copies measured in | HSV1 Copies measured in |
|---|---|---|
| Sample | 1 μl of extracted DNA | 50 μl of extracted DNA |
| HSV 10⁷ pfu | 81.67 | 4050 |
| HSV 10⁶ pfu | 26.68 | 1334 |
| HSV 10⁵ pfu | 5.13 | 256,5 |
| HSV 10⁴ pfu | 2.52 | 126 |
| HSV 10³ pfu | 3.48 | 174 |

### Example 3: Evaluation of Clinical Samples

The method was applied next on clinical samples.

The samples were analyzed for the detection of the following pathogens:
SARS-Cov-2 (at least 19 samples were analyzed).
Influenza B (at least 14 samples were analyzed).
Influenza A (at least 4 samples were analyzed).
RSV (at least 1 sample was analyzed).
Bordetella pertussis (Bp) (at least 3 samples were analyzed)

All the samples that were tested using the method of the invention showed 100% accuracy of the positive and the negative results, indicating 100% sensitivity and specificity of the detection method.

### Sars-Cov-2

Samples of patients with SARS-Cov-2 infection (clinical samples obtained from Shiba hospital and confirmed as SARS-Cov-2 positive by real time PCR performed at the hospital) were used for the preparation of RCA products. The RCA products were prepared as described in Example 1, using SARS-Cov-2 specific primers as shown in Table 1 (SEQ ID Nos 17 and 18), and a circular probe as shown in Table 2 (SEQ ID No. 21).

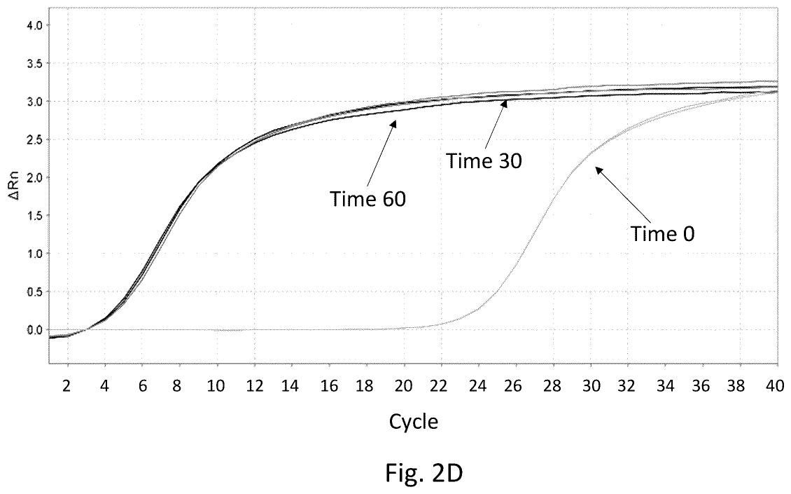

The RCA products were visualized on a gel. As can be seen in FIG. 4, the method was sensitive and specific allowing the detection of SARS-Cov-2 in clinical samples obtained from patients.

To account for potential background non-relevant signal, the Ct, and the melting temperature (TM) values were measured for the RCA products.

Table 6 shows the Ct and TM values for SARS-Cov-2 RCA products obtained from different clinical samples (I-IV), as compared with various controls.

TABLE 6
Ct and TM values

| Sample | Ct | Tm |
|---|---|---|
| SARS-Cov-2 (I) | 10.14 | 82.74 |
| SARS-Cov-2 (II) | 9.72 | 82.32 |
| SARS-Cov-2 (III) | 9.92 | 82.74 |
| SARS-Cov-2 (IV) | 9.98 | 82.74 |
| Non-specific | — | unspecific |
| gDNA | | Tm |
| sSARS-Cov-2 | 11.91 | 81.31 |
| sUU - negative | — | unspecific |
| | | Tm |

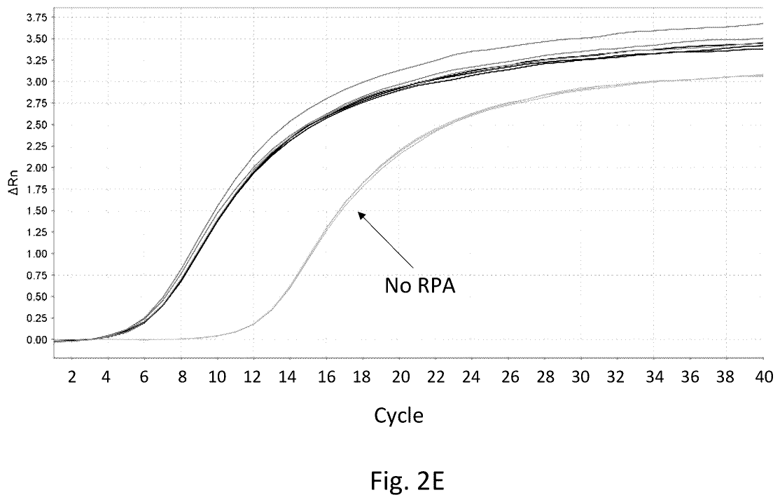

Clinical samples were obtained from Shiba (Sars-Cov-2), Hadassah (RSV) and Ichilov (infB). As can be seen in FIG. 5, the amplification process performed on clinical samples was sensitive, resulting in a clear identification of the target pathogen with a high signal to noise ratio clearly indicating the presence of RSV-B, influenza A or SARS-CoV2 in separate clinical samples.

### Example 4: Performing the Assay in a Cartridge

The amplification method of the invention can be performed in a specialized cartridge. As demonstrated in FIG. 6, the sample may be applied onto a cartridge in which all steps, including the isothermal amplification, are performed leading to an assay report providing the detection results. The steps that are performed in the cartridge comprise: nucleic acid (e.g., genomic DNA or RNA) extraction, pathogen selective nucleic acid isothermal amplification, circular probe hybridization with the target nucleic acid, ligation of positive circular probes, elimination of non-ligated probes and genomic DNA or RNA, RCA amplification of positive probes, cutting RCA products to short oligos, hybridization and detection of amplified targets with carbon array probes.

### Example 5: Preamplification Using Gp32-Mediated Homologous Recombination Followed by RCA

In this example, the detection efficacy of the method of the invention which combines Gp32 mediated homologous recombination (as a preamplification step) with RCA was compared to the detection efficacy of Gp32 mediated homologous recombination alone. The methods were tested as a multiplex with samples of Influenza A and Mycoplasma Pneumonia genomic DNA from Vircell.

### Materials and Methods

Lysis and extraction: These steps were performed as described above. Namely, 600 μl lysis solution (50% Solution A (2.5M Guanidinium Thiocyanate and 0.7% sodium N-lauroylsarcosinate in 0.1M Tris-HCl pH 6.5), 35% Isopropanol, 6 μl carrier RNA (1 mg/ml), 20 μl Speed Beads Magnetic Carboxylate, and DDW), and 40 μl Proteinase K were added to 200 μl of the clinical sample and vortexed for 30 sec. The mixture was then incubated for 2 minutes at 80° C. The tube was then transferred into a magnet holder and magnetized for 20 seconds until the beads were collected on the wall of the tube. Supernatant was discarded and the tube was removed from the magnet.

The extracted DNA was washed twice with a Wash buffer (Tris 66.8 nM, pH-7 KCl 330.3 mM), optionally further comprising up to 70% ethanol. Elution+preamplification: The Elution step is performed in a solution comprising 30 μL H₂O and 20 μL Gp32 pre-amplification buffer comprising: 50 mM Tris-Acetate (Sigma, T2694, pH 8.0), 7 mM Magnesium acetate (Sigma, 63052), 50 mM Potassium acetate (Sigma, 95843F), 2 mM DTT (Sigma, 1719757), 630 μM dNTPs (New England Biolabs: NEB, N0447L), Forward and Reverse primers each 1 μM, 0.33 units/μL Bsu large fragment (New England Biolabs: NEB, M0330L) or 0.33 units/μL Klenow exo-DNA polymerases (New England Biolabs: NEB, M0212L), 200 units/μL RevertAid Reverse Transcriptase (Thermo Scientific, EP0441), and 500 ng/μL T4-gp32 (New England Biolabs: NEB, M0300L).

After combining all the reagents together with the tested sample, reactions are incubated at 40° C. for 10 min.

In control samples the elution was performed in H₂O alone.

The ligation and the RCA steps were performed as described above.

Unlabeled DNA oligonucleotides (primers) of the multi-plex targets were purchased from Integrated DNA Technologies (IDT) and their sequences are listed in Table 7.

TABLE 7
Nomenclature and sequences of the DNA
primers for performing the multiplex
preamplification with Gp32 mediated
homologous recombination.

| | | SEQ |
|---|---|---|
| Name | Sequence (5′-3′) | ID NO |
| Fr_RPA_Chp | GTA AAG AAT AAC | 29 |
| | GAG CAC CAC GCT | |
| | CTC CAG CAA CC | |
| Rv_RPA_Chp | CAG GAG GAA TTA | 30 |
| | TGT GCA AAA CTC | |
| | TTA GCT CAA GG | |
| Fr_RPA_Lp | ATT GTT TAT GAA | 31 |
| | GAT GAA ATT GGT | |
| | GAC TGC GGC TG | |
| Rv_RPA_Lp | CCA ATG CTA TAA | 32 |
| | GAC AAC TTA TCC | |
| | TTG TCT GTA GC | |
| Fr_RPA_Mp | CAT TGG CTT GGG | 33 |
| | TTT GCA GCT CTT | |
| | GAT AAT GAG GG | |
| Rv_RPA_Mp | TGT CCG TAG TGC | 34 |
| | TTG ACT AGT AGA | |
| | TGC TGT TCC CG | |
| RSVA_RPA_Fr4_ | GCA CAC CAG CAG | 35 |
| CP15 + 17 | CAA ACA ATC GAG | |
| | CCA GAA G | |
| RSVA_RPA_Rv2_ | CGA TTG CAG ATC | 36 |
| 15 + 17 | CAA CTC CTA ACA | |
| | AAA AAC CAA G | |
| R1_hRSVB_HDA | CCT CTT CTT GCT | 37 |
| | TAT TGA TAC ATT | |
| | TAG G | |
| F2_hRSVB_HDA | CAA GAA TTA GAT | 38 |
| | AAG TAT AAG AAT | |
| | GCA GTA ACA G | |
| FluA RPA Fr 3 | GTT CTT TCT ATC | 39 |
| | ATC CCG TCA GGC | |
| | CCC CTC AAA GC | |
| FluA RPA Rv 2 | CCT AAA ATT CCC | 40 |
| | TTA GTC AGA GGT | |
| | GAC AAG ATT GG | |
| FluB RPA Fr2 | GAG ACA TCA AAG | 41 |
| | CCA AGA CTG CCT | |
| | ATG AAA AGA TTC | |
| FLuB RPA Rv2 | GCC ACA GAG GGC | 42 |
| | CTA ACA ACG ACC | |
| | ATA CTA CGA GC | |
| Bp_RPA_Fr3 | GAC GCT ACG GAC | 43 |
| | CTT CGC GAT GTG | |
| | TAG GGG TTG | |
| Bp_RPA_Rv3 | CTA CCA GAG CGA | 44 |
| | ATA TCT GGC ACA | |
| | CCG GCG CAT TC | |
| Covid19 40 bp | ACA ATG TAA CAC | 45 |
| RPA Fr 1 | AAG CTT TCG GCA | |
| | GAC | |
| Covid19 HDA | GTG CAA TTT GCG | 46 |
| REV 2 | GCC AAT GTT TGT | |
| | AAT C | |

### Influenza A (FluA)

The samples contained 5,000, or 1,000 copies of Influenza A gRNA from Vircell. The Gp32 mediated homologous recombination products (referred to herein as the “Gp32 HR products”) and the products of the combined methods (referred to herein as the “Gp32 HR+RCA products”) were prepared as described above, using Influenza A specific primers as shown in Table 7 (SEQ ID Nos 39 and 40), and a circular probe as shown in Table 2 (SEQ ID No. 22).

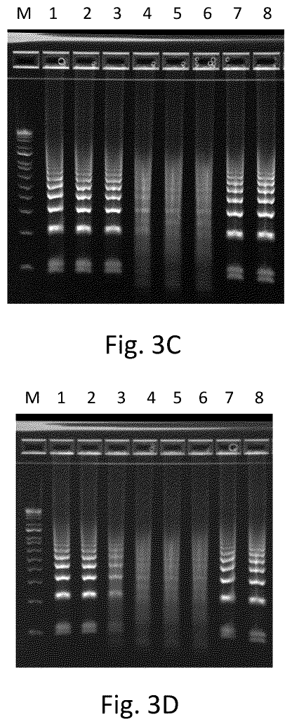

The Gp32 HR products and the Gp32 HR+RCA products were visualized on a gel (E-Gel® 2% Agarose, Thermo Fisher Scientific). As can be seen in FIG. 7, use of Gp32 mediated homologous recombination alone did not produce a detectable signal both for the sample containing 1000 copies and for the sample containing 5000 copies of FluA gRNA Vircell. In contrast, the method combining Gp32 mediated homologous recombination with RCA resulted in a clear detection signal, allowing the detection of as low as about 1000 copies of the pathogen per sample.

To test a possible limit of detection samples with a low copy number (i.e., 500, 100 and 50 copies of Influenza A) were assessed. The products of the combined methods (the “Gp32 HR+RCA products”) were prepared as described above, using Influenza A specific primers as shown in Table 7 (SEQ ID Nos 39 and 40), and a circular probe as shown in Table 2 (SEQ ID No. 22).

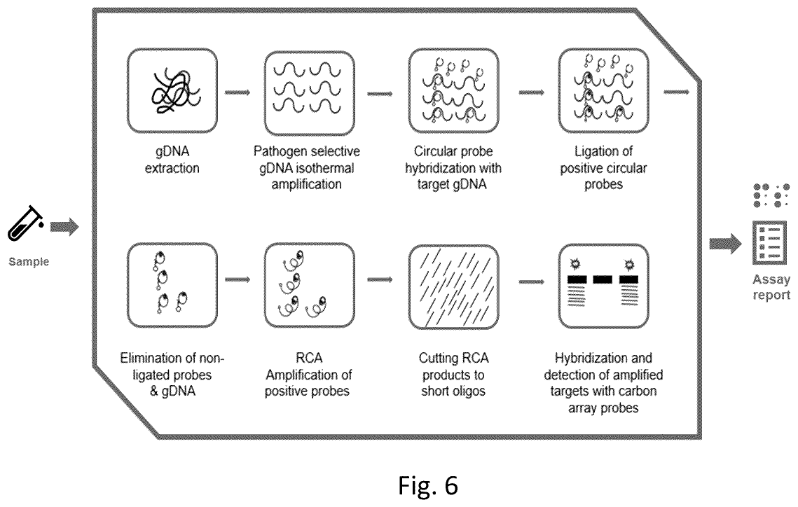

The Gp32 HR+RCA products were visualized on a gel (E-Gel® 2% Agarose, Thermo Fisher Scientific). As can be seen in FIG. 8, the method combining Gp32 mediated homologous recombination with RCA resulted in a clear detection signal, allowing the detection of as low as about 50 copies of the pathogen gRNA (Vircell) per sample. The clarity of the results indicates that the assay did not reach yet its limit of detection, which appears to be, for influenza A, far lower than 50 copies. This advantage is especially emphasized by the fact that the duration of the preamplification step is only 10 minutes.

Mycoplasma pneumoniae (Mp)

The samples contained 100, or 1,000 copies of Mycoplasma pneumoniae gDNA (Vircell). The Gp32 HR+RCA products and the RCA only (without prior amplification with Gp32 HR) products were prepared as described above, using Mycoplasma pneumoniae specific primers as shown in Table 7 (SEQ ID Nos 33 and 34), and a circular probe as shown in Table 2 (SEQ ID No. 26).

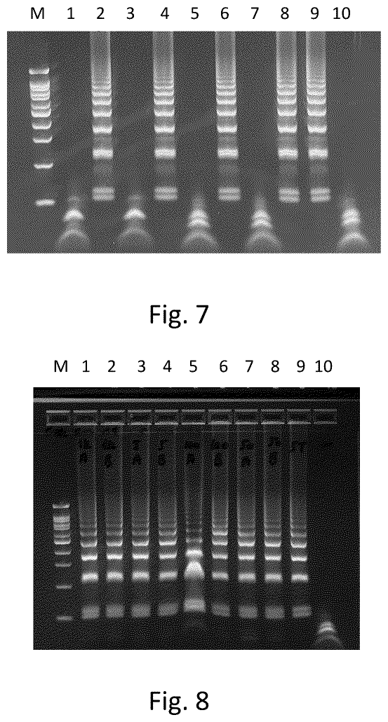

The Gp32 HR+RCA products and the RCA only products were visualized on a gel (E-Gel® 2% Agarose, Thermo Fisher Scientific). As can be seen in FIG. 9, use of RCA alone did not produce a detectable signal both for the sample containing 100 copies and for the sample containing 1000 copies of Mp. In contrast, the method combining Gp32 mediated homologous recombination with RCA resulted in a clear detection signal, allowing the detection of as low as about 100 copies of the pathogen gDNA (Vircell) per sample.

To account for potential background non-relevant signal, Tm values were measured for the qPCR of the RCA products. The Tm values were determined by the Quant Studio Design (Applied Biosystmes by Thermo Fisher Scientific) and its Analysis software according to the peak in the melting curve. Results revealed specific amplification for both 100 and 1,000 copies of Mp when the combined method of PA32 and RCA were performed. When only RCA was performed, no amplification was detected.

To summarize, the above presented results clearly indicate that the combined method of PA32 and RCA is advantageous over the use of RCA alone, namely the two amplification steps are essential for sensing/detecting low amounts of a pathogen gDNA (e.g., 100-5000 copies per sample) for which PA32 alone or RCA alone are not sufficient.

### Example 6: Preamplification Using Homologous Recombination Followed by RCA-Evaluation of Clinical Samples

Bordetella pertussis (Bp)

Clinical samples of Bp were collected from Shiba hospital. The samples were tested using the PA/RCA protocol as described above and were detected using sensors which were prepared for detection of 9 different targets: Influenza A (Flu A), Influenza B (Flu B), SARS Cov2 (covid19), RSV A, RSV B, Bortedella pertussis (Bp), Chlamydophila pneumoniae (Chp), Mycoplasma Pneumonia (Mp) and Legionella pneumophila (Lp).

### Results of the Sensor:

Three sensors, each sensor per sample recorded the true positive for the target Bp at signal to noise (S/N) of 5.4, 5.7 and 4.0. All negative spots reflected a value range of 0.6-1.2. The results are presented in Table 8 for each of the samples (Samples ID: #23233569, #150029779 and #23233531).

TABLE 8
Signal to noise values

| Array ID M100149B19 | Target | Signal | STD | CV | S/N |
|---|---|---|---|---|---|
| sample: 23233531 | Chp | 30.0 | 5.9 | 20% | 0.8 |
| | Bp | 196.2 | 8.9 | 5% | [symbol] |
| | Mp | 35.5 | 1.7 | 5% | 1.0 |
| | Lp | 42.3 | 10.3 | 24% | 1.2 |
| | RSVA | 42.2 | 16.6 | 39% | 1.2 |
| | FluA | 32.9 | 3.6 | 11% | 0.9 |
| | RSVB | 39.6 | 7.4 | 19% | 1.1 |
| | FluB | 32.9 | 1.6 | 5% | 0.9 |
| | CoV2 | 27.1 | 4.5 | 17% | 0.7 |
| Array ID M100149B18 | Target | Signal | STD | CV | S/N |
| sample # 150029779 | Chp | 26.8 | 4.0 | 15% | 0.8 |
| | Bp | 183.1 | 7.4 | 4% | [symbol] |
| | Mp | 28.6 | 1.9 | 7% | 0.9 |
| | Lp | 28.0 | 3.0 | 11% | 0.9 |
| | RSVA | 26.3 | 1.1 | 4% | 0.8 |
| | FluA | 28.9 | 2.1 | 7% | 0.9 |
| | RSVB | 29.7 | 1.6 | 6% | 0.9 |
| | FluB | 35.5 | 4.1 | 11% | 1.1 |
| | CoV2 | 37.0 | 5.7 | 16% | 1.1 |
| Array ID M100149B16 | Target | Signal | STD | CV | S/N |
| sample: #23233569 | Chp | 33.6 | 7.1 | 21% | 0.7 |
| | Bp | 180.6 | 7.5 | 4% | [symbol] |
| | Mp | 37.3 | 2.4 | 6% | 0.8 |
| | Lp | 36.2 | 9.3 | 26% | 0.8 |
| | RSVA | 34.2 | 13.1 | 38% | 0.8 |
| | FluA | 39.3 | 4.8 | 12% | 0.9 |
| | RSVB | 35.6 | 1.7 | 5% | 0.8 |
| | FluB | 45.3 | 5.3 | 12% | 1.0 |
| | CoV2 | 28.7 | 1.6 | 6% | 0.6 |

[symbol]

indicates data missing or illegible when filed

### Example 7: Preamplification Using BST DNA Polymerase Followed by RCA

In the following example, Bordetella pertussis (Bp) bacteria were identified using the dual amplification procedure whereby the amplicon was formed using BST2 or BST3 at a high temperature (60°-70° C.) followed by RCA.

Genomic DNA from 160000 copies of B. pertussis was purified and subjected to the following pre-amplification protocol:

Elution step: In the Elution step which includes primer insertion into the double helix DNA the eluted DNA was combined with a primer mix solution comprising H₂O, 0.8 μM of each Forward and Reverse primers, 0.2 μM of forward outer and reverse outer primers and 6% PEG 35K. The elution step is performed at 95° C. for 2 minutes followed by incubation at 55° C. for 1 minutes.

The following primers were used:

F_BP-ptxA
(SEQ ID NO: 56)
5′-AGTCTGCTGGCTGACGTAG-3′
Bp F3 (forward outer primer)
(SEQ ID NO: 57)
5′-CGACGCTACGGACCTTCG-3′
R_BP-ptxA
(SEQ ID NO: 58)
5′-AACATCCGCAGGGTAACGC-3′
Bp R3 (reverse outer primer)
(SEQ ID NO: 59)
5′-CGCTGGCCACCTACCA-3′

Pre-amplification step: the DNA/primers solution obtained in the elution step was incubated for 15 minutes at 62° C. in a solution comprising 1× Isothermal Buffer Pack II (New England Biolabs: NEB, B0374S), 1.4 mM each dNTPs (New England Biolabs: NEB, N0447L), 8 mM Magnesium sulfate (New England Biolabs: NEB, B1003S), and 0.16 units/μL Bst3 large fragment (New England Biolabs: NEB, M0374L). After combining all the reagents together with the DNA and primers, reactions are incubated at 65° C. for 15 min. In control samples the elution was performed in H₂O alone. An aliquot was taken at this stage for Bp-specific qPCR to evaluate the preamplification reaction efficiency. Table 9 shows the Ct and TM values for the Bp pre-amplification products, as compared with various controls. As indicated by the reduction in the Ct value, the preamplification step resulted in amplification of the target nucleic acid.

TABLE 9
Ct and TM values

| | Ct | Tm | ΔCt |
|---|---|---|---|
| Genomic Bp | 14.7 | 83.9 | 14.1 |
| DDW | 28.1 | 83.4 | 0.7 |
| Time “0” | 28.8 | 83.6 | |

Next, ligation and RCA steps were performed on the pre-amplified products as described above using a circular probe as shown in Table 2 (SEQ ID No. 20), and the final product was mounted on the sensor.

The sensor recorded the true positive for the target Bp at signal to noise of 7.4. All negative spots reflected a value range of 0.8 to 1. The results are presented in Table 10

TABLE 10
Signal to noise values

| Target | Signal | STD | CV | S/N |
|---|---|---|---|---|
| Chp | 23.6 | 7.7 | 0.3 | 1.0 |
| Bp | 175.1 | 27.2 | 0.2 | 7.4 |
| Mp | 18.7 | 2.6 | 0.1 | 0.8 |
| Lp | 19.4 | 5.2 | 0.3 | 0.8 |
| RSVA | 21.3 | 6.0 | 0.3 | 0.9 |
| FluA | 22.4 | 4.8 | 0.2 | 0.9 |
| RSVB | 22.8 | 2.5 | 0.1 | 0.9 |
| FluB | 21.4 | 10.0 | 0.5 | 0.9 |
| CoV2 | 19.6 | 4.5 | 0.2 | 0.8 |

1. A method for detection of a target nucleic acid in a sample, comprising:
a. said target nucleic acid into an amplicon;
b. amplifying said amplicon by performing a rolling circle amplification (RCA) to obtain an RCA product; and
c. said RCA product,
wherein a positive detection signal indicates the presence of said nucleic acid in the sample.

2. The method of claim 1, wherein said step of converting the target nucleic acid into an amplicon comprises isothermal amplification.

3. A method for detection of a target nucleic acid in a sample, comprising:
a. nucleic acids, wherein said nucleic acids were extracted and eluted from said sample;
b. target specific nucleic acid sequences by isothermal amplification with target specific primers;
c. the amplified target sequences obtained in step (b) with a sequence specific open circular probe and ligating the nick in the open circular probe;
d. a rolling circle amplification (RCA) to obtain RCA products; and
e. detecting said RCA product,
wherein a positive detection signal indicates the presence of the target nucleic acid in the sample.

4. (canceled)

5. (canceled)

6. (canceled)

7. (canceled)

8. (canceled)

9. (canceled)

10. (canceled)

11. (canceled)

12. The method of claim 1 wherein said isothermal amplification is recombinase polymerase amplification (RPA).

13. The method of claim 12, wherein said RPA is performed by incubating the eluted nucleic acids with at least one recombinase, at least one polymerase, and optionally at least one reverse transcriptase.

14. The method of claim 13, wherein said incubation is for between about 5 minutes and about 40 minutes, e.g., 20 minutes at a temperature of between about 37° C. and 65° C.

15. (canceled)

16. The method of claim 3, wherein said isothermal amplification is performed by homologous recombination (HR).

17. The method of claim 16 wherein said homologous recombination is performed using a strand displacing DNA polymerase.

18. The method of claim 17 wherein said homologous recombination is performed by incubating the extracted nucleic acids with at least one strand displacing DNA polymerase, at least one set of forward primer and reverse primer that hybridize to said target nucleic acid sequences, nucleoside triphosphates, magnesium sulfate, optionally at least one set of outer forward primer and outer reverse primer, and optionally at least one reverse transcriptase.

19. (canceled)

20. (canceled)

21. The method of claim 16 wherein said homologous recombination is performed using a strand displacing DNA polymerase together with bacteriophage T4 Gp32.

22. The method of claim 21 wherein said homologous recombination is performed by incubating the extracted nucleic acids with bacteriophage T4 Gp32, at least one DNA polymerase, at least one set of forward primer and reverse primer that hybridize to said target nucleic acid sequences, nucleoside triphosphates, magnesium acetate, optionally potassium acetate and optionally at least one reverse transcriptase.

23. The method of claim 22, wherein said incubation is for between about 5 minutes and about 15 minutes, e.g., 10 minutes at a temperature of between about 37° C. and 42° C., e.g., 40° C.

24. The method of claim 16, wherein said at least one set of forward primer and reverse primer that hybridize to said target nucleic acid sequences are pathogen specific primers selected from a group consisting of SEQ ID Nos: 29-46.

25. The method of claim 3 wherein said sequence specific open circular probe is a pathogen specific probe selected from a group consisting of SEQ ID Nos: 20-28, and 53-55.

26. The method of claims 1 wherein the method further comprises a step of exonuclease treatment after ligating the nick in the open circular probe and prior to performing the RCA.

27. The method of claim 1 wherein said RCA is performed using branching forward primers and branching reverse primers.

28. The method of claim 1 wherein said RCA is performed in the presence of nickase.

29. (canceled)

30. (canceled)

31. (canceled)

32. (canceled)

33. A cartridge for detecting at least one pathogen in a sample, comprising buffers, solutions, primers, probes, and enzymes for performing:
a. Pathogen lysis;
b. Nucleic acid purification;
c. Target amplification;
d. Ligation of a circular probe with the amplified target;
e. RCA;
f. Digestion; and
g. Detection.

34. A kit for detecting at least one pathogen in a sample, comprising:
a. a cartridge according to claim 33; and
b. for use.

35. A system for detecting at least one pathogen in a sample, comprising:
a. a benchtop analyzer; and
b. at least one cartridge according to claim 33.


SEQUENCE LISTING (59 sequences)

SEQ ID NO: 1 - DNA, 35 bp, synthetic construct
  source 1..35: mol_type = other DNA
gtaaagaataacgagcaccacgctctccagcaacc

SEQ ID NO: 2 - DNA, 35 bp, synthetic construct
  source 1..35: mol_type = other DNA
caggaggaattatgtgcaaaactcttagctcaagg

SEQ ID NO: 3 - DNA, 35 bp, synthetic construct
  source 1..35: mol_type = other DNA
attgtttatgaagatgaaattggtgactgcggctg

SEQ ID NO: 4 - DNA, 35 bp, synthetic construct
  source 1..35: mol_type = other DNA
ccaatgctataagacaacttatccttgtctgtagc

SEQ ID NO: 5 - DNA, 35 bp, synthetic construct
  source 1..35: mol_type = other DNA
cattggcttgggtttgcagctcttgataatgaggg

SEQ ID NO: 6 - DNA, 35 bp, synthetic construct
  source 1..35: mol_type = other DNA
tgtccgtagtgcttgactagtagatgctgttcccg

SEQ ID NO: 7 - DNA, 26 bp, synthetic construct
  source 1..26: mol_type = other DNA
ttatgaattatacactcaacaatgcc

SEQ ID NO: 8 - DNA, 19 bp, synthetic construct
  source 1..19: mol_type = other DNA
gcaatgccactggcgattg

SEQ ID NO: 9 - DNA, 28 bp, synthetic construct
  source 1..28: mol_type = other DNA
cctcttcttgcttattgatacatttagg

SEQ ID NO: 10 - DNA, 34 bp, synthetic construct
  source 1..34: mol_type = other DNA
caagaattagataagtataagaatgcagtaacag

SEQ ID NO: 11 - DNA, 35 bp, synthetic construct
  source 1..35: mol_type = other DNA
gttctttctatcatcccgtcaggccccctcaaagc

SEQ ID NO: 12 - DNA, 35 bp, synthetic construct
  source 1..35: mol_type = other DNA
cctaaaattcccttagtcagaggtgacaagattgg

SEQ ID NO: 13 - DNA, 36 bp, synthetic construct
  source 1..36: mol_type = other DNA
gagacatcaaagccaagactgcctatgaaaagattc

SEQ ID NO: 14 - DNA, 35 bp, synthetic construct
  source 1..35: mol_type = other DNA
gccacagagggcctaacaacgaccatactacgagc

SEQ ID NO: 15 - DNA, 35 bp, synthetic construct
  source 1..35: mol_type = other DNA
tggcgcgagtctgctggctgacgtagcgagcgttg

SEQ ID NO: 16 - DNA, 35 bp, synthetic construct
  source 1..35: mol_type = other DNA
ttccgcccgaaaacatccgcagggtaacgcgggtc

SEQ ID NO: 17 - DNA, 27 bp, synthetic construct
  source 1..27: mol_type = other DNA
acaatgtaacacaagctttcggcagac

SEQ ID NO: 18 - DNA, 28 bp, synthetic construct
  source 1..28: mol_type = other DNA
gtgcaatttgcggccaatgtttgtaatc

SEQ ID NO: 19 - DNA, 47 bp, synthetic construct
  source 1..47: mol_type = other DNA
tctctctctctctctctctctctcgagtcgctaccttgccctaaacg

SEQ ID NO: 20 - DNA, 117 bp, synthetic construct
  source 1..117: mol_type = other DNA
tcgccggtgatgccgttgtgaggcgtttagggcaaggtagcgactcttttagccgttcctcggcccagacttttcaacagacaggaacaggcttttgaatactccgtggtcgtggtc

SEQ ID NO: 21 - DNA, 123 bp, synthetic construct
  source 1..123: mol_type = other DNA
tggactttattgacactaagaggggaggcgtttagggcaaggtagcgactcttttagccgttcctcggcccagacttttcaacagacaggaacaggctttttcatgcactttgtccgaacaac

SEQ ID NO: 22 - DNA, 120 bp, synthetic construct
  source 1..120: mol_type = other DNA
gtctttgcaggaaagaacacagataggcgtttagggcaaggtagcgactcttttagccgttcctcggcccagacttttcaacagacaggaacaggctttttcgcgcagagactggaaagt

SEQ ID NO: 23 - DNA, 116 bp, synthetic construct
  source 1..116: mol_type = other DNA
agttgatcaagtgatcggaaaggcgtttagggcaaggtagcgactcttttagccgttcctcggcccagacttttcaacagacaggaacaggcttttgcgccccaacaaaaggctct

SEQ ID NO: 24 - DNA, 111 bp, synthetic construct
  source 1..111: mol_type = other DNA
tcttggttttttgttaggaggcgtttagggcaaggtagcgactcttttagccgttcctcggcccagacttttcaacagacaggaacaggcttttaaaaggaaaagaagatt

SEQ ID NO: 25 - DNA, 118 bp, synthetic construct
  source 1..118: mol_type = other DNA
accacagtatatgaactacacaggcgtttagggcaaggtagcgactcttttagccgttcctcggcccagacttttcaacagacaggaacaggcttttcaaccgggctagaagagaagc

SEQ ID NO: 26 - DNA, 115 bp, synthetic construct
  source 1..115: mol_type = other DNA
catgaccaggttcaacgggaacaggcgtttagggcaaggtagcgactcttttagccgttcctcggcccagacttttcaacagacaggaacaggcttttaccagccgggtggtgag

SEQ ID NO: 27 - DNA, 118 bp, synthetic construct
  source 1..118: mol_type = other DNA
aacgagatacagacgactgggaggcgtttagggcaaggtagcgactcttttagccgttcctcggcccagacttttcaacagacaggaacaggcttttcagcctgaatctttcgtagcc

SEQ ID NO: 28 - DNA, 118 bp, synthetic construct
  source 1..118: mol_type = other DNA
cattgcaagccccataacagcaggcgtttagggcaaggtagcgactcttttagccgttcctcggcccagacttttcaacagacaggaacaggcttttggttgcagccattgctgttga

SEQ ID NO: 29 - DNA, 35 bp, synthetic construct; identical to SEQ ID NO: 1
  source 1..35: mol_type = other DNA

SEQ ID NO: 30 - DNA, 35 bp, synthetic construct; identical to SEQ ID NO: 2
  source 1..35: mol_type = other DNA

SEQ ID NO: 31 - DNA, 35 bp, synthetic construct; identical to SEQ ID NO: 3
  source 1..35: mol_type = other DNA

SEQ ID NO: 32 - DNA, 35 bp, synthetic construct; identical to SEQ ID NO: 4
  source 1..35: mol_type = other DNA

SEQ ID NO: 33 - DNA, 35 bp, synthetic construct; identical to SEQ ID NO: 5
  source 1..35: mol_type = other DNA

SEQ ID NO: 34 - DNA, 35 bp, synthetic construct; identical to SEQ ID NO: 6
  source 1..35: mol_type = other DNA

SEQ ID NO: 35 - DNA, 31 bp, synthetic construct
  source 1..31: mol_type = other DNA
gcacaccagcagcaaacaatcgagccagaag

SEQ ID NO: 36 - DNA, 34 bp, synthetic construct
  source 1..34: mol_type = other DNA
cgattgcagatccaactcctaacaaaaaaccaag

SEQ ID NO: 37 - DNA, 28 bp, synthetic construct
  source 1..28: mol_type = other DNA
cctcttcttgcttattgatacatttagg

SEQ ID NO: 38 - DNA, 34 bp, synthetic construct; identical to SEQ ID NO: 10
  source 1..34: mol_type = other DNA

SEQ ID NO: 39 - DNA, 35 bp, synthetic construct; identical to SEQ ID NO: 11
  source 1..35: mol_type = other DNA

SEQ ID NO: 40 - DNA, 35 bp, synthetic construct; identical to SEQ ID NO: 12
  source 1..35: mol_type = other DNA

SEQ ID NO: 41 - DNA, 36 bp, synthetic construct; identical to SEQ ID NO: 13
  source 1..36: mol_type = other DNA

SEQ ID NO: 42 - DNA, 35 bp, synthetic construct; identical to SEQ ID NO: 14
  source 1..35: mol_type = other DNA

SEQ ID NO: 43 - DNA, 33 bp, synthetic construct
  source 1..33: mol_type = other DNA
gacgctacggaccttcgcgatgtgtaggggttg

SEQ ID NO: 44 - DNA, 35 bp, synthetic construct
  source 1..35: mol_type = other DNA
ctaccagagcgaatatctggcacaccggcgcattc

SEQ ID NO: 45 - DNA, 27 bp, synthetic construct
  source 1..27: mol_type = other DNA
acaatgtaacacaagctttcggcagac

SEQ ID NO: 46 - DNA, 28 bp, synthetic construct
  source 1..28: mol_type = other DNA
gtgcaatttgcggccaatgtttgtaatc

SEQ ID NO: 47 - DNA, 25 bp, synthetic construct
  source 1..25: mol_type = other DNA
ggcaaacactaataccgatttcaag

SEQ ID NO: 48 - DNA, 24 bp, synthetic construct
  source 1..24: mol_type = other DNA
gcttttacttgcaagacattcctc

SEQ ID NO: 49 - DNA, 25 bp, synthetic construct
  source 1..25: mol_type = other DNA
cagaatttggcgtacctcaatttcg

SEQ ID NO: 50 - DNA, 25 bp, synthetic construct
  source 1..25: mol_type = other DNA
tgtggaatattggatatggcctgac

SEQ ID NO: 51 - DNA, 25 bp, synthetic construct
  source 1..25: mol_type = other DNA
ccacagggtcagctcgtgattctgc

SEQ ID NO: 52 - DNA, 24 bp, synthetic construct
  source 1..24: mol_type = other DNA
atcaagaccacctcctccatcgag

SEQ ID NO: 53 - DNA, 116 bp, synthetic construct
  source 1..116: mol_type = other DNA
ctaggacgcagtgccgccagaggcgtttagggcaaggtagcgactcttttagccgttcctcggcccagacttttcaacagacaggaacaggcttttgacatcttgtctggctttaa

SEQ ID NO: 54 - DNA, 112 bp, synthetic construct
  source 1..112: mol_type = other DNA
cggaaccgacgcacaatgaggcgtttagggcaaggtagcgactcttttagccgttcctcggcccagacttttcaacagacaggaacaggcttttgatttgattttcgccatc

SEQ ID NO: 55 - DNA, 116 bp, synthetic construct
  source 1..116: mol_type = other DNA
catgtcaacgatatgttgggaggcgtttagggcaaggtagcgactcttttagccgttcctcggcccagacttttcaacagacaggaacaggcttttcgtacaaccacatacagcgc

SEQ ID NO: 56 - DNA, 19 bp, synthetic construct
  source 1..19: mol_type = other DNA
agtctgctggctgacgtag

SEQ ID NO: 57 - DNA, 18 bp, synthetic construct
  source 1..18: mol_type = other DNA
cgacgctacggaccttcg

SEQ ID NO: 58 - DNA, 19 bp, synthetic construct
  source 1..19: mol_type = other DNA
aacatccgcagggtaacgc

SEQ ID NO: 59 - DNA, 16 bp, synthetic construct
  source 1..16: mol_type = other DNA
cgctggccacctacca